(19) United States
(12) Patent Application Publication
(10) Pub. No.: US 2007/0190153 A1
(43) Pub. Date: Aug. 16, 2007

(54) Delivery systems for non-steroidal anti-inflammatory drugs (nsaids)

(76) Inventor: Jonathan Farber, Montreal (CA)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 10/591,789

(22) PCT Filed: Oct. 29, 2004
(86) PCT No.: PCT/CA04/01895

§ 371 Date: Mar. 6, 2007

Related U.S. Application Data
(60) Provisional application No. 60/550,603, filed on Mar. 5, 2004.

(51) Int. Cl. A61K 31/405; A61K 31/192; A61K 9/14

(52) U.S. Cl. 424/488; 514/563; 514/569; 514/630; 514/420

(57) ABSTRACT

Oral gel delivery systems for non-steroidal anti-inflammatory drugs (NSAIDs) are provided comprising an ingestible matrix within which one or more NSAIDs are substantially uniformly and completely dispersed. The delivery systems may optionally include one or more other functional ingredients that complement or enhance the function of the NSAID(s) within the body.

### CROSS REFERENCE TO RELATED APPLICATIONS

This application is a continuation-in-part of U.S. patent application Ser. No. 10/416,547, filed Mar. 25, 2003, which is a national stage of PCT application PCT/CA03/00411, filed Mar. 25, 2003. The aforesaid PCT application claims priority from U.S. Provisional Patent Application Ser. No. 60/372,438, filed Apr. 16, 2002. The contents of all of the aforementioned applications are hereby specifically incorporated by reference in their entirety.

### FIELD OF THE INVENTION

The present invention pertains to the field of oral delivery systems, in particular to a gel delivery system for non-steroidal anti-inflammatory drugs.

### BACKGROUND OF THE INVENTION

Non-steroidal anti-inflammatory drugs (NSAIDs) have been in use for over a century beginning with aspirin. NSAIDs are widely administered for their analgesic and/or anti-inflammatory and/or anti-pyretic effects and are used in the alleviation of pain and inflammation in a variety of situations, including pain and inflammation associated with rheumatoid arthritis, osteoarthritis, juvenile arthritis, ankylosing spondylitis, tendinitis, bursitis, and gout.

Pharmaceutically active agents can be administered to the patient in many forms with oral administration being the most popular. Pharmaceutical dosage forms intended for oral administration can be provided as liquid solutions, emulsions, suspensions or in solid form as tablets, capsules, pills, lozenges or caplets. Such dosage forms have traditionally been used for the administration of NSAIDs. Various liquid NSAID formulations have been described. For example, U.S. Pat. No. 4,684,666 describes a stabilized ibuprofen syrup comprising ibuprofen or a pharmacologically acceptable salt or ester thereof suspended in an aqueous liquid having greater than 50% by weight of a polyhydric alcohol bodying agent, a sweetening agent, a stabilizing agent, and an antioxidant. The syrup is formulated to be higher than pH 7.0 and lower than pH 7.5.

European Patent No. 0,896,815 describes a suspension for an acidic sparingly soluble drug, such as ibuprofen. The suspension has a pH value between 2 and 5. The suspension comprises the drug with particle size from 1 to 15 microns, a polyglycerol fatty acid ester, a water soluble polyhydric alcohol and water.

U.S. Pat. No. 5,079,001 describes a liquid suspension for diclofenac with a pH between about 2.0 and about 3.5. The suspension may contain preservatives, antoxidants, suspending agents, wetting agents, as well as fragrances, dyes and sweeteners.

In cases where the dosage to be administered cannot be made into a very small tablet or capsule, or in cases where children, older persons and many other persons are unable to swallow whole tablets and capsules, soft gelatine capsules (softgels—the currently accepted nomenclature adopted by the SoftGel Association) and chewable dosage forms are used. A softgel is a one-piece, hermetically sealed soft gelatine shell containing a liquid, a suspension, or a semi-solid. Soft gelatine capsules serve chiefly for the containment of liquids, i.e. oily solutions, suspensions or emulsions. Vegetable, animal and mineral oils, liquid hydrocarbons, ethereal oils and also polyethylene glycols are in use as fillings. Fats and waxes are also applied or admixed to increase the consistency.

A number of NSAID formulations suitable for delivery in a softgel format have been described. For example, U.S. Pat. No. 5,468,502 describes a solution of ibuprofen suitable for filling soft gelatine capsules. The solution comprises at least about 25% by weight of ibuprofen, about 1% to 10% by weight of water, about 50% to 74% of a solubilizing material selected from non-ionic polyethoxylated surface active agents alone or in combination with a solvent system and about 1% to 10% ammonium acetate. The solvent system includes alcohols, polyols and fatty acid esters having 2 to 21 carbon atoms.

U.S. Patent Application 2003/0219477 describes formulations of NSAIDs for filling soft gelatine capsules. The solvent system for the NSAIDs comprises 40% to 60% by weight of polyoxyethylene ether, 15% to 35% by weight of glycerol and 15% to 35% by weight of water. The formulations further comprise an effective amount of sodium or potassium hydroxide.

Solvents suitable for human consumption, however, such as ethanol, propylene glycol, dimethyl acetamide, lactic acid, glycerol, and butanediol, have been shown to be unsuitable for introduction into soft gelatine capsules in larger quantities because the capsule fillings made with these solvents cause, after a short time, softening and deformation of the capsules produced, which therefore are not marketable.

Gelatine has also been used to prepare sustained release tablets. For example, U.S. Pat. No. 6,068,854 describes a sustained release medicament tablet comprising gelatine and/or fractionated gelatine and a lipophilic or poorly water soluble pharmaceutical substance. Preparation of the tablet comprises compressing a powdered gelatine pharmaceutical substance mixture. The mixture may be prepared by physical mixing of the components or by spray-drying a gelatine solution to which the pharmaceutical substance has been added.

Chewable systems are also employed in the administration of pharmaceutical active agents. Palatability and “mouth feel” are important characteristics to be considered in providing a chewable dosage form for a pharmaceutical. The palatability of the chewable dosage form can be a critical factor in ensuring patient compliance. Many pharmaceuticals and other active ingredients have a bitter or otherwise unpalatable taste, or an unacceptable mouth-feel, due to the grittiness or chalkiness of the compound, or both. As a result, incorporation of such active ingredients into standard chewable dosage forms can lead to difficulties in obtaining compliance by the user due to the objectionable taste and/or mouth feel of the product.

Several approaches have been used to overcome these problems. The poor taste of a pharmaceutical or other active ingredient may be masked by using suitable flavouring compounds and/or sweeteners. Coating with fats or oils or encapsulation of the active ingredient may also serve to mask bitterness and other undesirable tastes. For example, U.S. Pat. No. 5,489,436 describes chewable tablets made from a coated medicament where the coating is a “reverse enteric coating” designed to be soluble at the acidic pH of the stomach but relatively insoluble in the mouth. The coatings comprise a polymer blend of dimethylaminoethyl methacrylate and neutral methacrylic acid ester and a cellulose ester.

U.S. Pat. No. 6,136,347 describes taste-masked microcapsules for use in liquid suspension formulations, particularly in oil-based juices or a suitable liquid such as water. The microcapsule comprises an active ingredient granule coated with a single outer polymeric coating derived from film-forming agents such as neutral methyl and ester compounds of polymethacrylic acid. The coatings are designed to be water-insoluble and rapidly degrade once the composition reaches the acidic environment of the stomach.

Other techniques for providing a chewable delivery system involve the use of a gum base. Gum bases are insoluble elastomers which form the essential element for chewing gum. A coating containing the active ingredient is then applied over the confectionery gum. As the dosage form is chewed, the coating fractures and/or is dissolved in the mouth and swallowed. This approach is currently employed with gum-based products manufactured by Schering Plough HealthCare, such as aspirin (Aspergum™) and U.S. Pat. No. 6,613,346 describes a chewing gum centre including a compressible powder that is compressed around the centre. The powder includes a medicament that may or may not be encapsulated. Dosage forms of this nature (especially aspirin) may not provide the active ingredient as a bioavailable agent to the same extent as an oral tablet dosage form (see “Relative Bioavailability of Aspirin Gum,” J. Pharm. Sci., 70:1341 (1981)).

Other chewable delivery systems have been described. For example, European Patent No. 0 336 894 describes a sugarless gel confectionery system comprising hydrogenated starch hydrolysates, pectin algin, a polymer network gel and an edible insoluble solid structuring component. The system may contain up to 20% humectant. The final solids content of 80-90% in the product is achieved by boiling off excess moisture.

U.S. Pat. No. 5,637,313 describes a soft chewable dosage form including a matrix comprising hydrogenated starch hydrolysates, a water soluble bulking agent and a water insoluble bulking agent. The matrix is formed under high shear at room temperature and contains minor amounts of humectant such as glycerol or glycol.

U.S. Patent Application 2003/0228368 describes an edible composition as a dosage form which comprises between 25% to 40% of a non-aqueous carrier with a melting point below 45° C. and a thermoplastic material with a melting point greater than 50° C. and optionally up to 40% by weight of a material for retaining the non-aqueous carrier in the composition.

International Patent Application No. PCT/US97/20217 (WO 98/20860) describes a hydrocolloid based delivery system comprising a sweetener, a hydrocolloid and water, having a solids content between about 50% and about 83%. Preservatives are added to the delivery system when the solids content is less than 78%.

U.S. Pat. No. 6,432,442 describes a chewable composition comprising a matrix comprising gelatine and hydroxypropyl cellulose capable of being chewed and swallowed in less than about 20 seconds. Coated or encapsulated actives are added directly to the assembled matrix.

U.S. Pat. No. 4,882,154 describes a more shelf-stable gelatine-based chewable delivery system. This system, however, requires the use of pre-coated drugs, vitamins and minerals in order to preserve the stability of these compounds.

International Patent Applications WO 03/026438, WO 03/026439 and WO 03/088755 describe gel-like delivery systems for creatine and other functional ingredients. The delivery systems described by these applications comprise as essential components a carbohydrate (such as a starch) and at least one hydrocolloid component (such as gelatine or a plant gum).

Other chewable delivery systems for minerals and other functional ingredients include troches (or lozenges), which are a traditional drug dosage format that is based on gelatine and glycerine and are used in preparing custom medications by hand for individual patients. Troches are made in small quantities from a base that typically comprises 70% glycerine, 10% gelatine and 20% water. The water is slowly driven off by heating the base and the final composition, which tends to absorb moisture from the air, is stored under refrigeration. The troche itself is made by re-melting the base and adding milligram quantities of an active ingredient. Troches are not stable and are intended to be consumed within thirty days. Typically, methyl paraben is included in the base material to prevent microbial spoilage.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide a delivery system for non-steroidal anti-inflammatory drugs (NSAIDS). In accordance with an aspect of the present invention, there is provided an oral gel delivery system for non-steroidal anti-inflammatory drugs (NSAIDs) comprising one or more NSAIDs substantially uniformly dispersed in a gel matrix, said delivery system having a final moisture content of between about 10% and about 40% by weight and a water activity of less than about 0.9, and said gel matrix comprising: (a) one or more hydrocolloids; (b) one or more sugars, sugar syrups, sugar alcohols, or a combination thereof; and (c) one or more polyhydric alcohols.

In accordance with another aspect of the invention, there is provided an oral gel delivery system for non-steroidal anti-inflammatory drugs (NSAIDs) comprising one or more NSAIDs substantially uniformly dispersed in a gel matrix, said delivery system having a final moisture content of between about 10% and about 30% by weight and a water activity of less than about 0.7, and said gel matrix comprising: (a) one or more hydrocolloids selected from the group of: modified starch, gelatine, gellan, pectin, cellulose and modified cellulose; (b) one or more sugar syrups selected from the group of: corn syrup, high fructose corn syrup, maltitol syrup and isomalt syrup, and (c) one or more polyhydric alcohols selected from the group of: glycerol and propylene glycol.

In accordance with another aspect, the oral gel delivery system of the present invention further comprises one or more other functional ingredients, wherein the total amount of said one or more NSAIDs and said one or more functional ingredients is less than or equal to 40% by weight of said delivery system.

In accordance with another aspect of the invention, there is provided a use of a gel matrix comprising: (a) one or more hydrocolloids; (b) one or more sugars, sugar syrups, sugar alcohols, or a combination thereof, and (c) one or more polyhydric alcohols, in the preparation of an oral gel delivery system for non-steroidal anti-inflammatory drugs (NSAIDs), wherein said delivery system comprises one or more NSAIDs substantially uniformly dispersed in said gel matrix, and said delivery system has a final moisture content of between about 10% and about 40% by weight and a water activity of less than about 0.9.

In accordance with another aspect of the invention, there is provided a process for preparing an oral gel delivery system for non-steroidal anti-inflammatory drugs (NSAIDs), said process comprising the steps of: (i) preparing a blend of one or more hydrocolloids, one or more sugars, sugar syrups, sugar alcohols, or a combination thereof, and optionally water at a temperature of less than 100° C., wherein said hydrocolloid(s), said sugars, sugar syrups and/or sugar alcohols and said water are in a ratio that will provide a final moisture content to the delivery system of between about 10% and about 40% by weight; (ii) reducing the temperature of said blend to between about 50° C. and about 80° C.; (iii) dispersing one or more NSAIDs in a solvent comprising one or more polyhydric alcohols at a temperature at or below about 70° C. to provide a solvent mixture; (iv) combining said blend from step (ii) with said solvent mixture to provide a gel matrix, and (v) moulding said gel matrix to provide said oral gel delivery system.

In accordance with another aspect of the invention, there is provided an oral gel delivery system for non-steroidal anti-inflammatory drugs (NSAIDs) prepared by the above-described process.

In accordance with another aspect, there is provided a use of an oral gel delivery system of the invention to deliver an effective amount of one or more non-steroidal anti-inflammatory drugs (NSAIDS) to an animal in need thereof.

In accordance with another aspect, there is provided a kit for the delivery of one or more non-steroidal anti-inflammatory drugs (NSAIDs) to an animal comprising one or more units of an oral gel delivery system of the invention and optionally instructions for use.

### BRIEF DESCRIPTION OF THE FIGURES

FIG. 1 demonstrates absorption of a functional ingredient into the blood following administration of a delivery system prepared with a gel matrix according to one embodiment of the invention.

### DETAILED DESCRIPTION OF THE INVENTION

Unless defined otherwise, all technical and scientific terms used herein have the same meaning as commonly understood by one of ordinary skill in the art to which this invention pertains. As used herein, percentage values (%) represent the weight percentages of the total weight of the delivery system.

The term “functional ingredient,” as used herein, includes physiologically or pharmacologically active substances intended for use in the treatment, prevention, diagnosis, cure or mitigation of disease or illness, or that provide some degree of nutritional, physiological or therapeutic benefit to an animal when consumed. The term refers more particularly to a substance that affects beneficially one or more target functions in the body, in a way that is either an improved state of health or well-being and/or reduction of risk of disease. Non-limiting examples include drugs, botanical extracts, enzymes, hormones, proteins, polypeptides, antigens, nutritional supplements such as fatty acids, antioxidants, vitamins, minerals, as well as other pharmaceutically or therapeutically useful compounds. A functional ingredient in the context of the present invention refers to an ingredient included in the delivery system of the invention in addition to those ingredients that constitute the gel matrix itself. In the context of the present invention, a NSAID is a functional ingredient.

The terms “non-steroidal anti-inflammatory drugs” or “NSAID” or “NSAIDs,” as interchangeably used herein, refer to aniline derivatives, propionic acid derivatives, acetic acid derivatives, fenamic acid derivatives, biphenylcarboxylic acid derivatives, salicylic acid derivatives, pyrazolone derivatives and oxicams, Cox-2 inhibitors and pharmaceutically acceptable salts, esters, isomers, and mixtures thereof. The term also encompasses pro-drug forms of the above compounds.

The term “nutritional supplement,” as used herein, refers to a substance that exerts a physiological effect on an animal. Typically, nutritional supplements fulfil a specific physiological function or promote the health or well-being of the consumer.

The terms “botanical extract” and “botanical,” as used interchangeably herein, refer to a substance derived from a/plant source. Non-limiting examples include echinacea, Siberian ginseng, ginko biloba, kola nut, goldenseal, golo kola, schizandra, elderberry, St. Johns Wort, valerian, ephedra and the like.

The term “drug,” as used herein, refers to a pharmacologically active substance that exerts a localised or systemic effect or effects on an animal.

The term “pro-drug,” as used herein, refers to an inactive precursor of a drug that has to be metabolised or otherwise processed in vivo following administration in order to exhibit pharmacologic activity.

The term “treatment,” as used herein, refers to an intervention performed with the intention of improving a patient's status. The improvement can be subjective or objective and is related to the alleviation of the symptoms associated with a condition being treated.

The term “alleviate” or “alleviation” includes the arrest, decrease, or improvement in one or more the symptoms, signs, and features of the condition being treated, both temporary and long-term.

The terms “subject” and “patient” as used herein refer to an animal in need of treatment.

The term “animal,” as used herein, includes, but is not limited to, mammals (including humans), birds and reptiles.

As used herein, the term “about” refers to a +/−10% variation from the nominal value. It is to be understood that such a variation is always included in any given value provided herein, whether or not it is specifically referred to.

NSAID Delivery Systems

The NSAID delivery systems of the present invention are gel delivery systems that comprise one or more NSAIDs dispersed in an ingestible matrix. The delivery system may further comprise one or more other functional ingredients that complement or enhance the function of the NSAID(s) within the body. The matrix of the delivery system provides for substantially uniform and complete dispersion of the NSAID(s) (and other functional ingredients) and helps to minimise degradation of heat labile functional ingredients during manufacturing. The matrix of the delivery system further provides for minimised degradation of the functional ingredients during subsequent storage of the final delivery system. The NSAID delivery systems are suitable for administration to an animal, for example, in order to alleviate pain, reduce inflammation or reduce fever, or a combination thereof.

The delivery systems of the present invention comprise one or more NSAID (and optionally other functional ingredients) substantially uniformly dispersed within a gel matrix which comprises 1) one or more hydrocolloids; 2) a sugar component and 3) a solvent component. The selection of appropriate hydrocolloid(s) as described herein in amounts within the ranges indicated results in a matrix that readily retains the solvent component and thereby helps to prevent separation of the solvent from other components of the matrix. Additives, such as natural or artificial flavourings, colourings, acidulants, buffers and sweeteners can be included in conventional amounts in the matrix. The matrix may also include one or more sources of monovalent cations or divalent cations, if required, to allow for proper set-up of the matrix. If insufficient water is provided by the various components selected to formulate the matrix, additional water may be added to the matrix as necessary to provide the desired final moisture content within the range indicated below.

The delivery systems may further comprise one or more compounds that act to enhance the bioavailability of the NSAID(s) and other functional ingredients (i.e. “bioavailability enhancers”), as discussed in more detail below.

Due to the substantially uniform and complete dispersion of the NSAID(s) within the matrix, the delivery systems of the invention are suitable for division into sub-units. For example, if a single unit of a delivery system is divided into three subunits, each subunit will contain a third of the dose of the original unit. Such division would not be possible with other delivery systems in which the functional ingredients are not evenly dispersed.

As indicated above, the matrix of the delivery systems provides for minimised degradation of functional ingredients during the preparation of the matrix and the storage of the final delivery systems. The use of relatively low temperatures in the preparation of the matrix, when compared to typical manufacturing procedures for confectioneries, ensures that the functional ingredients are not degraded by excessive heat. In accordance with the present invention, the functional ingredients are added to the other components of the matrix to prepare the delivery system at a temperature of 100° C. or less. In one embodiment of the present invention, the entire preparation process takes place at or below 100° C. In another embodiment, the delivery systems are prepared at or below a temperature of 75° C. In another embodiment, the delivery systems are prepared at or below a temperature of 70° C. In a further embodiment, the delivery systems are prepared at or below a temperature of 65° C. Low temperatures can be employed in the preparation of the delivery system because the matrix is formulated to be flowable at low temperatures by selection of appropriate ingredients as described herein. In one embodiment of the invention, the matrix is flowable at or above 45° C. In another embodiment, the matrix is flowable at or above 35° C.

The delivery systems of the present invention are intermediate moisture products and maintain a low interaction with water during and after preparation of the matrix, which can also contribute to the stability of some of the functional ingredients dispersed therein. Although the actual amount of moisture and final water activity (a_{w}) of an intermediate moisture food has not been defined precisely, general opinion is that an intermediate moisture product should have a moisture content between about 10% and about 40% by weight and an a_{w} below about 0.9 (see, S. Hegenbart, “Exploring Dimensions in Intermediate Moisture Foods,” (1993) Food Product Design, Weeks Publishing Company, Northbrook, Ill.). In accordance with the present invention, therefore, the final moisture content of the delivery systems is between about 10% and about 40%. In one embodiment, the final moisture content of the delivery systems is between about 10% and about 30%. In another embodiment, the final moisture content of the delivery systems is between about 11% and about 25%. In other embodiments, the moisture content is between about 13% and about 20%, and between about 14% and about 18%.

In addition, the delivery systems of the present invention have an a_{w} below about 0.9. In one embodiment of the invention, the water activity of the final delivery systems is below about 0.85. In another embodiment, the water activity of the final delivery systems is below about 0.8. In a further embodiment, the water activity is below about 0.7. In another embodiment, the water activity is below about 0.6. Alternatively, the water activity of the final delivery systems may be described as being between about 0.45 and about 0.7. In one embodiment, the water activity is between about 0.5 and about 0.6.

For those functional ingredients that are susceptible to degradation, for example, due to heat lability; degradation during the process of preparing the matrix of the delivery systems is minimised. In one embodiment, degradation of the functional ingredients during preparation of the matrix is less than about 20%. In another embodiment, degradation of the functional ingredients during preparation of the matrix is less than about 15%. In other embodiments, degradation of the functional ingredients during preparation is less than about 10%, less than about 5%, less than about 3% and less than about 2%.

Degradation of the functional ingredients during storage of the final delivery systems under normal storage conditions (i.e. at temperatures of 30° C. or below) is also minimised. In accordance with the present invention, therefore, degradation of the functional ingredients during storage of the delivery systems under normal conditions is less than about 20%. In one embodiment, degradation of the functional ingredients during storage is less than about 15%. In other embodiments, degradation of the functional ingredients during storage is less than about 10%, less than about 5%, less than about 3% and less than about 2%.

The matrix to be used in the delivery systems of the invention can be formulated to have a final pH in the range of about 2.5 to about 9.0. As will be appreciated by one skilled in the art, however, selection of the final pH for the matrix will be influenced by the properties of the functional ingredients to be included in the final delivery system. Thus, for the NSAID delivery systems of the invention, the matrix is formulated such that the delivery systems have a final pH in the range of about 4.5 to about 9.0. In one embodiment, the matrix is formulated such that the delivery systems have a final pH in the range of about 5.0 to about 9.0. In another embodiment, the matrix is formulated such that the delivery systems have a final pH in the range of about 5.5 to about 9.0. In another embodiment, the matrix is formulated such that the delivery systems have a final pH in the range of about 6.0 to about 9.0. In further embodiments, the matrix is formulated such that the delivery systems have a final pH in the range of about 6.0 to about 8.5 and about 6.5 to about 8.5.

In their final form, the delivery systems of the present invention are semi-solid, intermediate moisture systems, having some properties clearly identified with those of jellies and some properties that are similar to the jujube variety of confectioneries. In the context of the present invention, the term “semi-solid” indicates that the delivery system has properties that, depending on the measurement, are a mixture of solid and liquid behaviours. The matrix of the delivery systems, therefore, is formulated to be semi-solid at normal room temperature. In the event, however, that the matrix liquefies due to exposure to elevated temperatures, the formulation of the matrix is such that no phase separation of the components occurs and the matrix can be readily re-solidified by cooling (for example, by cooling to temperatures of around 4° C.). The reformed product maintains the substantially uniform dispersion of the NSAID(s) (and other optional functional ingredients) contained therein. In one embodiment of the present invention, the delivery systems are formulated such that the matrix is a semi-solid at temperatures at or below about 40° C. In another embodiment, the delivery systems are semi-solid at or below about 35° C. In other embodiments, the delivery systems are semi-solid at or below about 30° C. and at or below about 25° C.

The gel delivery systems according to the present invention are suitable for administration to both human and non-human animals. One skilled in the art will appreciate that each delivery system can be formulated differently according to the type of animal to which it is to be administered. For example, for administration to an animal such as a cat or a dog, meat or fish-based flavours may be added. For administration to a human, the delivery system may be formulated, for example, as a confectionery using fruit-based or other confectionery flavours. The delivery systems are especially suited for oral administration due to their palatability. Additionally, due to the highly portable format, the delivery systems are simple and convenient to administer and to consume for both humans and other animals.

The texture, physical attributes, form and shape of the matrix as described below, can be varied by altering the ratio of ingredients within the given ranges using the methods described herein or by methods familiar to a worker skilled in the art.

1. The Matrix

As indicated above, the delivery systems of the invention comprise one or more NSAIDs dispersed in a matrix that comprises 1) one or more hydrocolloids; 2) a sugar component and 3) a solvent component. For the purposes of the present invention, “hydrocolloids” can be divided into carbohydrate-based hydrocolloids and non-carbohydrate based hydrocolloids. The delivery system of the present invention can comprise one or more carbohydrate-based hydrocolloids, one or more non-carbohydrate based hydrocolloids, or a combination of one or more carbohydrate-based hydrocolloids with one or more non-carbohydrate based hydrocolloids.

1.1 Hydrocolloid

The matrix according to the present invention comprises one or more hydrocolloids that perform the functions of water binding and gelation and contribute to the overall texture and body of the gel matrix. Hydrocolloids can also be used to improve and/or stabilise the texture of a food product while inhibiting crystallisation.

Hydrocolloids are hydrophilic polymers of vegetable, animal, microbial or synthetic origin. Non-carbohydrate based hydrocolloids are typically animal-derived, a representative example being gelatine (hydrolysed collagen). Carbohydrate-based hydrocolloids are typically plant derived and include starches (and other amylaceous ingredients) and polysaccharide-based gums. An “amylaceous ingredient” as used herein refers to a food-stuff that contains a preponderance of starch and/or starch-like material. Examples of amylaceous ingredients include cereal grains and meals or flours obtained upon grinding cereal grains such as corn, oats, wheat, milo, barley, rice, as well as the various milling by-products of these cereal grains such as wheat feed flour, wheat middlings, mixed feed, wheat shorts, wheat red dog, oat groats, hominy feed, and other such material. Other sources of amylaceous ingredients include tuberous foodstuffs, such as potatoes, tapioca, and the like.

Suitable starches for use in the delivery systems are typically modified starches derived from a variety of plant sources such as, for example, corn, waxy corn, wheat, rice, tapioca, potato, pea and other sources known in the art. Modified starches are known in the art refer to starches that have been physically or chemically altered to improve their bioactive characteristics. Suitable modified starches include, but are not limited to, pre-gelatinised starches, low viscosity starches (such as dextrins, acid-modified starches, oxidized starches and enzyme modified starches), derivatised starches, stabilised starches (such as starch esters and starch ethers), cross-linked starches, starch sugars (such as glucose syrup, dextrose and isoglucose) and starches that have been submitted to a combination of treatments (such as cross-linking and gelatinisation) and mixtures thereof.

Examples of suitable polysaccharide-based gums that can be used in the delivery systems include, but are not limited to, Konjac, tragacanth gum, guar gum, acacia gum, karaya gum, locust bean gum, xanthan gum, agar, pectin, carageenan, gellan , alginate, and various cellulose gums. Suitable cellulose gums for use in the preparation of the matrix are typically modified cellulose gums including, for example, methylcellulose (MC), hydroxypropyl methylcellulose (HPMC), ethyl cellulose (EC), hydroxyethyl cellulose (HEC), hydroxypropylcellulose (HPC), hydroxypropyl methylcellulose acetate, hydroxyethyl methylcellulose, hydroxyethylcellulose acetate, hydroxyethyl ethylcellulose and combinations thereof.

The use of hydrocolloids is well-known in the art and many hydrocolloids for use in products for human or animal consumption are available commercially, for example, gelatines from Leiner Davis, various polysaccharide gums and blends manufactured by CP Kelco, the Ticagel® range of hydrocolloids from TIC Gums, modified starches from A.E. Staley and a range of modified celluloses known as Methocel Food Gums manufactured by Dow Chemical Company.

In one embodiment of the present invention, the gel matrix comprises gelatine. Gelatine is defined generally using a “Bloom value” which indicates the strength of the gel formed under certain circumstances using the gelatine. In the preparation of confectionery, when a harder gel is desired, gelatine having a higher Bloom value is used. Conversely, when the final product is required to be more flowing, gelatine having a lower Bloom value is used. One skilled in the art will appreciate that the water holding capacity of gelatine alone is lower than that of a combination of gelatine with another hydrocolloid, such as gellan or pectin. Thus, the use of gelatine alone as the hydrocolloid in the delivery system may necessitate the use of a higher amount of gelatine to achieve the desired gelation/texture of the matrix, than when gelatine is used in combination with one or more other hydrocolloids. When the hydrocolloid in the matrix of the present invention comprises gelatine, the Bloom value (BL) is generally about 100 to 260 BL. Combinations of gelatines with different Bloom values also can be used. The gelatine can be derived from a variety of sources, for example, beef, pork, chicken or fish gelatine (or a combination thereof) may be used.

When the gel matrix comprises gelatine, the gelatine can be combined with one or more other hydrocolloids to impart different characteristics to the matrix. For example, combinations of gelatine with gellan or gelatine with pectin provide a good texture to the matrix. Addition of a modified starch to one of these combinations also provides textural improvements.

When combinations of gelatine and gellan or pectin are used in the preparation of the matrix, the ratio of gelatine:gellan or gelatine:pectin is typically in the range between about 15:1 to about 40:1. These relative amounts provide a cohesive structure to the delivery system.

Similarly, a combination of a modified starch with one or more other hydrocolloids can impart certain desirable features to the matrix, for example, modified starch can contribute to the structural integrity of the matrix and its low set temperature. It can also provide heat stability to the finished product as well as the ability to bind a limited quantity of fats/oils if required.

The use of combinations of modified starches and modified celluloses as the hydrocolloid component of the matrix is also contemplated by the present invention as discussed below in Section 1.5.

An example of a suitable type of modified starch for inclusion in the matrix is one that is able to fully hydrate and develop its viscosity in the presence of the other matrix-forming components at a temperature below 100° C., for example at a temperature of, or below, 70° C. Such starches are often referred to as “low set temperature” starches. While the majority of carbohydrates hydrate upon heating, certain starches, which are commercially available and are known in the art as “cold set” or “pre-gelatinised” starches are capable of hydrating at room temperature and are also suitable for use in the gel matrix.

One skilled in the art will appreciate that the viscosity development of the selected hydrocolloid or hydrocolloid mixture should allow for sufficient ease of mechanical handling and pumping during production as well as allowing sufficient time to incorporate all the ingredients and to mould the final product before it sets.

In addition, it will be understood that the hydrocolloid(s) to be used in the gel matrix will depend on the desired final pH of the matrix, the particular texture and consistency required for the final product and, if more than one hydrocolloid is used, the interaction of the hydrocolloids. Certain combinations of hydrocolloids are known in the art to provide synergistic effects, for example, the combination of xanthan (which does not gel well alone) with Konjac, or carageenan and Konjac.

The type of hydrocolloid, or mixture of hydrocolloids, used can also affect the set temperature of the matrix. For example, the use of a gelatine/gellan mixture or a gelatine/pectin mixture provides a set temperature around 35° C., whereas the use of carageenan or locust bean gum will result in a set temperature closer to 60° C. Thus, the choice of hydrocolloid(s) for use in the matrix is also dependent upon the properties of the functional ingredient(s) to be incorporated into the delivery system. Functional ingredients that are unstable at higher temperatures will require the selection of a hydrocolloid or mixture of hydrocolloids that have a low set temperature, whereas functional ingredients that are more stable can be used with hydrocolloid(s) having a higher set temperature.

The use of hydrocolloids in intermediate moisture products is well known in the art and a skilled technician would readily be able to select an appropriate hydrocolloid or mixture of hydrocolloids for use in the delivery systems of the invention. In one embodiment of the present invention, the delivery system comprises one or more modified starch, alone or in combination with one or more other hydrocolloid. Non-limiting examples of hydrocolloids suitable for use with modified starch include gelatine; gellan and gelatine; pectin and gelatine; gellan, gelatine and one or more cellulose or modified cellulose; and pectin, gelatine and one or more cellulose or modified cellulose. In another embodiment of the present invention, the delivery system comprises gelatine, alone or in combination with one or more other hydrocolloid. Non-limiting examples of hydrocolloids suitable for use with gelatine include one or more modified starch; gellan; pectin; cellulose or modified cellulose; gellan and one or more modified starch; pectin and one or more modified starch; gellan and one or more cellulose or modified cellulose; pectin and one or more cellulose or modified cellulose; gellan, one or more modified starch and one or more cellulose or modified cellulose; and pectin, one or more modified starch and one or more cellulose or modified cellulose. In a further embodiment of the present invention, the delivery system comprises pectin in combination with one or more other hydrocolloid. Non-limiting examples of hydrocolloids suitable for use with pectin include gelatine; gelatine and one or more modified starch; gelatine and one or more cellulose or modified cellulose; and gelatine, one or more modified starch and one or more cellulose or modified cellulose.

The total amount of hydrocolloid(s) incorporated into the matrix is generally between about 0.1% and about 17% by weight. In one embodiment, the total amount of hydrocolloid(s) in the matrix is between about 0.6% to about 17% by weight. In a further embodiment, the total amount is between about 0.6% and about 15% by weight. In another embodiment, the total amount is between about 0.5% and about 10% by weight.

The selection of the actual amount of hydrocolloid(s) from within the ranges provided above to be included in the matrix will be dependent upon the type of hydrocolloid(s) being used and on the desired texture of the final product. Determination of this amount is considered to be within the ordinary skills of a worker in the art.

In one embodiment of the invention, the matrix comprises one or more modified starch in an amount between about 0.5% and about 10.0% by weight, for example, between about 1.7% and about 8.0%. In another embodiment, the matrix comprises gelatine in an amount between about 0.1% and about 10% by weight, for example between about 1.0% and 9.0%. In a further embodiment, the matrix comprises a polysaccharide-based gum in an amount between about 0.1% and about 5.0% by weight, for example, between about 0.2% and about 2.0%. In still another embodiment, the matrix comprises one or more modified cellulose in an amount between about 0.1% and about 3% by weight, for example, between about 0.6% and 1.5%.

In a specific embodiment of the invention, the matrix comprises a combination of one or more modified starch in an amount between about 0.5% and about 10.0% by weight, gelatine in an amount between about 0.1% and about 10.0% by weight, and a polysaccharide-based gum in an amount between about 0.1% and about 2.0% by weight.

1.2 Sugar Component

Sugar is generally used in a confection primarily for sweetness; however, it is known in the art that sugar can also play an important role in the physical properties of a matrix, such as crystallinity, gel strength, bodying/texture, humectancy, and water activity.

The sugar component of the matrix comprises one or more sugars, sugar syrups, sugar alcohols and/or sugar alcohol solids. Examples include, but are not limited to, sugars such as sucrose, glucose, xylose, ribose, maltose, galactose, dextrose, and fructose; syrups such as corn syrups, hydrogenated glucose syrups, high fructose corn syrups; polydextrose; and sugar alcohols such as isomalt, maltitol, sorbitol, lactitol and mannitol. The latter are also often in the form of syrups. One skilled in the art will appreciate that if a sugar or sugar alcohol solid is used in the matrix, it should be first dissolved, for example, by heating in water or in another syrup, prior to being added to the mixture.

When the sugar component comprises dextrose, it is generally provided in the form of a corn syrup. Corn syrups are prepared by hydrolysis of starch and are characterised by dextrose equivalent (D.E.) values such that they are classified as low, medium or high D.E. syrups, with high D.E. syrups having a high concentration of dextrose and low D.E. syrups having a low concentration of dextrose. In one embodiment of the present invention, the sugar component used in the preparation of the matrix comprises a corn syrup and/or a high fructose corn syrup. Suitable corn syrups are typically those with a D.E. between 20 D.E. and 99 D.E., for example, between about 40 D.E. and 70 D.E.

Various corn syrups are commercially available. For example, 62 D.E. 1600 Corn Syrup (Casco Inc./Canada Starch Operating Co. Inc.), SWEETOSE 4300 corn syrup (a 63 D. E. corn syrup; A. E. Staley Manufacturing Company; Decatur, Ill.) and Clearsweet® 63/43 IX corn syrup (a 63 D. E. corn syrup; Cargill/North America Sweeteners).

Combinations of sugars or sugar syrups are also suitable for use in the preparation of the matrix. Examples of suitable combinations of syrups include, but are not limited to, isomalt syrup and high fructose corn syrup, a high D.E. corn syrup and high fructose corn syrup and maltitol syrup and high fructose corn syrup.

One skilled in the art will appreciate that the total amount of the sugar component in the matrix will vary depending upon the type(s) of sugar used. For example, when sugar syrups are used, lower viscosity sugar syrups will produce a matrix with less body and lower rigidity. The total amount of the sugar component present in the matrix is about 10% to about 60% by weight.

In one embodiment of the present invention, the sugar component comprises a mixture of sugar syrups. In another embodiment, the sugar component comprises a mixture of sugar syrups in a total amount of between about 15% and about 55% by weight of the delivery system. In a further embodiment, the sugar component comprises a mixture of sugar syrups in a total amount between about 25% and about 55% by weight of the delivery system.

1.3 Solvent Component

The primary role of the solvent component of the matrix is to dissolve or disperse the functional ingredients to allow for substantially uniform and complete incorporation of these ingredients into the matrix. The solvent also provides for improved flow characteristics of the mixture and functions somewhat as a humectant. In accordance with one embodiment of the present invention, the NSAID(s) and/or other functional ingredients are added to the solvent component prior to combining with the remaining components of the matrix.

The solvent used in the preparation of the matrix is typically colourless and non-volatile with no strong odour or flavour and is substantially miscible with water and/or alcohols. In accordance with the present invention, the solvent component comprises one or more polyhydric alcohol. The term “polyhydric” as used herein means that the compound contains two or more hydroxyl groups. Examples of suitable polyhydric alcohols include, but are not limited to, glycerol and/or its lower alkyl ester derivatives, propylene glycol, and short chain polyallcylene glycols, such as polyethylene glycol, and mixtures thereof. As will be apparent to one skilled in the art, certain polyhydric alcohols may also function somewhat as sweeteners.

In one embodiment of the present invention, the solvent component comprises glycerol. In another embodiment, the solvent component comprises a mixture of glycerol and a short chain polyalkylene glycol. In a further embodiment, the solvent component comprises a mixture of glycerol and propylene glycol.

Typically, the delivery system according to the present invention contains about 5% to about 50% by weight of the solvent component. In one embodiment, the delivery system contains about 5% to about 38% by weight of the solvent component. In an alternate embodiment, the delivery system contains about 10% to about 50% by weight of the solvent component. In a further embodiment, the delivery system contains about 20% to about 48% by weight of the solvent component. In other embodiments, the delivery system contains between about 15% and about 50%, between about 15% and about 40% and between about 15% and 35% by weight of the solvent component.

1.4 Water

As indicated above, the delivery system according to the present invention has a final moisture content between about 10% and about 40% and a water activity below about 0.9. In one embodiment, the final moisture content of the delivery system is between about 10% and about 30% and the water activity is below about 0.7. It will be readily apparent to one skilled in the art that the appropriate amount of water may be provided by one or more of the various components of the system, for example, a sugar syrup, a hydrated starch or a hydrated hydrocolloid, or additional water may need to be added separately. Additional water can be provided alone or as a solution containing other additives, for example, as a buffer solution or as a solution containing a sweetener, flavouring or colouring. The total amount of water from the one or more sources will be sufficient to provide the final delivery system with a moisture content and water activity within the ranges indicated above.

1.5 Other Additives

The gel matrix can optionally contain other additives such as flavourings, colourings, additional sweeteners, modified vegetable gums or celluloses, mono- or divalent cations, or a combination thereof It will be readily apparent that additives for inclusion in the matrix should be selected such that they do not affect the properties of the matrix, do not exhibit substantial reactivity with the functional ingredients in the matrix, and are stable during preparation of the matrix.

One or more additional sweeteners can be selected from a wide variety of suitable materials known in the art. Representative, but non-limiting, examples of sweeteners include xylose, ribose, sucrose, mannose, galactose, fructose, dextrose, maltose, partially hydrolysed starch, lactose, maltodextrins, hydrogenated starch hydrolysate and mixtures thereof. In addition to these sweeteners, polyhydric alcohols such as sorbitol, mannitol, xylitol, and the like may also be incorporated. Alternatively, an artificial sweetener or a blend of artificial sweeteners can be used. Examples of suitable artificial sweeteners include, for example, sucrose derivatives (such as Sucralose), amino acid based sweeteners, dipeptide sweeteners, saccharin and salts thereof, acesulfame salts (such as acesulfame potassium), cyclamates, steviosides, dihydrochalcone compounds, thaumatin (talin), glycyrrhizin, aspartame, neotame, alitame, and mixtures thereof.

When an additional sweetener is used, it can be used in amounts as low as 0.01% by weight. The actual amount of sweetener required will be dependent on the type of sweetener selected and on the desired sweetness of the final product. Amounts of various sweeteners to be added to food products are well known in the art. When a natural sweetener is used, the total amount of the sugar component, which forms a structural part of the matrix, and additional sweetener(s) in the matrix, however, remains less than 60% by weight. In one embodiment of the invention, the matrix comprises one or more additional sweeteners. In another embodiment, the matrix comprises one or more artificial sweeteners.

Suitable flavourings that can be added to the delivery system are known in the art and include, both synthetic flavour oils and oils derived from various sources, such as plants, leaves, flowers, fruits, nuts, and the like. Representative flavour oils include spearmint oil, peppermint oil, cinnamon oil, and oil of wintergreen (methylsalicylate). Other useful oils include, for example, artificial, natural or synthetic fruit flavors such as citrus oils including lemon, orange, grape, lime and grapefruit, and fruit essences including apple, strawberry, cherry, pineapple, banana, raspberry and others that are familiar to a worker skilled in the art. A wide variety of synthetic flavourings suitable for inclusion in the matrix are known in the art and are commercially available. The amount of flavouring agent employed is normally a matter of preference subject to such factors as concentration/dilution of the flavour stock, flavour type, base type and strength desired. In general, amounts of about 0.01% to about 5.0% by weight of a final product are useful.

Colourings suitable for use in foodstuffs are well known in the art and can be optionally included in the matrix to add aesthetic appeal. A wide variety of suitable food colourings are available commercially, for example, from Warner Jenkins, St. Louis, Mo. Where a synthetic colouring agent is used in the matrix, the amount ranges from about 0.01% to about 2% by weight. A worker skilled in the art will appreciate that when a colouring agent derived from a natural source is used in the matrix, an increased amount of the colouring agent is generally required to achieve the same effect as a synthetic colouring agent.

The present invention also contemplates that modified vegetable gums or modified or unmodified celluloses may be included in the matrix in order to improve the texture, body, lubricity and/or elasticity of the matrix. These compounds can be used, for example, to increase the viscosity of the delivery system if it is warmed, thus reducing potential melting and lessening water activity which will help to improve the stability of the system in the event it is left in an excessively hot environment. Examples of modified vegetable gums or modified celluloses are provided above. Unmodified celluloses are also contemplated and are known in the art. Examples of cellulose include Solka-Flo® from International Fibre Corporation, North Tonawanda, N.Y., and powdered Avicel® microcrystalline cellulose from FMC Biopolymers, Philadelphia, Pa. Modified vegetable gums can be included in the matrix in amounts between about 0.01% and 2.0% by weight, for example between about 0.1% and about 1.5%. Modified or unmodified celluloses, or mixtures thereof, can be included in the matrix in amounts between about 0.1% and about 10.0% by weight, for example, between about 0.6% and about 5.0%.

If necessary, the matrix can also comprise one or more sources of monovalent cations and/or divalent cations to help facilitate gelation of the matrix. Suitable sources of mono- and divalent cations for incorporation into food products are known in the art and are commercially available. Non-limiting examples include mono- or divalent salts, such as sodium or potassium chloride and potassium citrate. Mono- or divalent salts can be added to the matrix, if required, in an amount between, for example, about 1% and about 5% by weight.

2. Non-Steroidal Anti-Inflammatory Drugs (NSAIDs)

As indicated above, the delivery systems of the present invention comprise one or more NSAIDs. A wide variety of NSAIDs are known in the art and are suitable for incorporation in the delivery systems (see, for example, “Goodman and Gilman's: The Pharmacological Basis of Therapeutics, ” Eds. Goodman, Limbird, Milinoff, Ruddon, Gilman & Hardman, McGraw-Hill Professional; 9th edition, 1996; “Remington: The Science and Practice of Pharmacy” Gennaro, A., Lippincott, Williams & Wilkins, Philidelphia, Pa., 2000).

Examples of suitable NSAIDs include, but are not limited to, aniline derivatives, e.g. acetaminophen, phenacetin; propionic acid derivatives, e.g. ibuprofen and stereoisomers thereof, naproxen, ketoprofen and the like; acetic acid derivatives, e.g. indomethacin, diclofenac, sulindac, tolmetin, and the like; fenamic acid derivatives, e.g. mefenamic acid, meclofenamic acid, flufenamic acid, and the like; biphenylcarboxylic acid derivatives, e.g. diflunisal, flufenisal, and the like; salicylic acid derivatives e.g. aspirin (acetyl salicylic acid), aalsalate, sodium salicylate, choline salicylate, choline magnesium salicylate, “buffered aspirin,” chitosan acetyl salicylic acid and the like; pyrazolone derivatives e.g. azapropazone, oxyphenbutazone, phenylbutazone and the like; and oxicams, e.g. piroxicam, sudoxicam, isoxicam, meloxicam, and the like; Cox-2 inhibitors e.g. Nimesulide, Meloxicam, Celecoxib, Rofecoxib and the like; and pharmaceutically acceptable salts, esters and isomers thereof.

The present invention also contemplates the use of pro-drug forms of NSAIDS. Pro-drugs constitute an inactive form of the NSAID that, upon in vivo administration, is metabolised or otherwise converted to the active form of the drug. Pro-drugs are typically designed such that the metabolic stability and/or transport characteristics of the drug are altered, the side effects or toxicity are reduced or the flavour of the drug is improved. Sulindac (Clinoril®) is a commercially available pro-drug form of an acetic acid derivative NSAID. Chitosan acetyl salicylic acid and chitosan oligosaccharide acetyl salicylic acid (commercially available from Oligopharm Co. Ltd., Nizhni Novgorod, Russia) can also be considered to be pro-drugs. In these compounds, the chitosan/chitosan oligosaccharide is ionically associated with acetyl salicylic acid and, upon dissociation in the stomach, releases active acetyl salicylic acid. The chitosan/chitosan oligosaccharide component is believed to provide some degree of gastroprotection. Chitosan can also act to increase absorption of the drug. Other examples of NSAID pro-drugs contemplated by the present invention are discussed in European Patent No. 0 331 471.

In one embodiment of the present invention, the delivery system comprises one or more aniline derivative NSAIDs, propionic acid derivative NSAIDs, acetic acid derivative NSAIDs or salicylic acid derivative NSAIDs, or a combination thereof. In another embodiment, the delivery system comprises an aniline derivative NSAID, propionic acid derivative NSAID or acetic acid derivative NSAID. In a further embodiment, the delivery system comprises a salicylic acid derivative NSAID. In another embodiment, the delivery system comprises acetyl salicylic acid, or a pharmaceutically acceptable salt, ester, isomer, buffered or pro-drug version thereof.

The delivery systems of the present invention are capable of incorporating up to 40% by weight of the selected NSAID(s). It will be readily apparent to a worker skilled in the art however, that based on typical dosages of NSAID(s), the delivery systems generally will incorporate less than 40% by weight of the NSAID(s). In one embodiment, the delivery system incorporates between about 0.2% and about 10% by weight of the selected NSAID(s). In another embodiment, the delivery system incorporates between about 0.4% and about 10% by weight of the selected NSAID(s).

3. Other Functional Ingredients

The present invention contemplates that additional functional ingredients that complement or enhance the function of an NSAID within the body may be added to the delivery systems. The delivery systems of the invention can be used in a variety of situations, for example, in alleviating pain and inflammation associated with injuries, arthritis, rheumatism, surgical procedures and the like, and in reducing fever and/or pain associated with colds, influenza and other infections. It is contemplated, therefore, that other functional ingredients known to contribute to the alleviation of a patient's symptoms in these situations may also be included in the delivery systems.

NSAIDs can also be employed in a prophylactic capacity, for example, in situations where the aim is to prevent or delay the occurrence of inflammation and/or minimise recovery time, either from an existing injury or as a result of strenuous activity. Prophylactic use of NSAIDs prior to surgery has also been shown to be beneficial. Thus the delivery systems of the present invention can comprise combinations of NSAID(s) with performance enhancing functional ingredients for use in enhancing an individual's endurance, performance or recovery, or with functional ingredients intended to reduce the effects of, or recovery time from, surgery.

The other functional ingredients included in the delivery systems can be, for example, drugs, therapeutic compounds, nutritional supplements, botanicals or herbal extracts, and the like, where use of such compounds is not contra-indicated. The selection of appropriate and compatible combinations of functional ingredients can be made readily by the skilled technician. As is known in the art, certain combinations of functional ingredients are incompatible due to undesirable interactions between the ingredients, for example, interactions that alter absorption, renal elimination, or hepatic metabolism of one or more of the functional ingredients, or that result in additive effects or toxicities. Accordingly, selection of appropriate combinations of functional ingredients can be made by the skilled worker based on knowledge in the art and publicly available information regarding contraindications of certain combinations (see, for example, The A-Z Guide to Drug-Herb and Vitamin Interactions, Schuyler W. Lininger (ed.) (1999) Three Rivers Press (Calif.);. Mosby's Handbook of Drug-Herb & Drug-Supplement Interactions, R. Harkness & S. Bratman (2002), Mosby; and the Mayo Clinic website).

A variety of drugs or therapeutic compounds are suitable for use with the delivery system of the invention. Representative examples include, but are not limited to, anti-inflammatory compounds such as steroids; anti-hypertensive drugs, vasoconstrictors, sedatives, antihistamines, decongestants, expectorants, anti-tussives, other analgesic compounds such as narcotic analgesics, alkaloids, muscle-relaxants, anaesthetics, antacids, anticholinergics/antispasmodics and anti-nauseants. Illustrative, but non-limiting, examples of nutritional supplements suitable for use with the delivery system of the invention include, probiotics, prebiotics, vitamins, enzymes, co-enzymes, cofactors, antioxidants, minerals and mineral salts, phytochemicals, phospholipids, other trace nutrients, botanical extracts, oat beta-glucan and other functional fibres, bicarbonate, citrate, or combinations thereof.

Exemplary anti-inflammatory compounds suitable for incorporation into the delivery systems of the invention include, but are not limited to, betamethasone, budesonide, cortisone, dexamethasone, hydrocortisone, methylprednisolone, prednisolone, prednisone and triamcinolone.

Exemplary antihistamines suitable for incorporation into the delivery systems of the invention include, but are not limited to, acrivastine, azatadine, brompheniramine, carbinoxamine, chlorpheniramine, clemastine, dexbrompheniramine, diphenhydramine, loratadine, pheniramine, phenyltoloxamine, promethazine, pyrilamine, and triprolidine. The effective dosage levels are compound specific and are known in the art. Typical dosages range between about 1.25 mg and about 50 mg.

Exemplary decongestants include, but are not limited to, phenylephrine and pseudoephedrine. Typical dosages range between about 5 mg and about 60 mg. Anti-tussives can be narcotics such as codeine, dihydrocodeine, hydrocodone and hydromorphone, or non-narcotics such as carbetapentane, caramiphen and dextromethorphan. Typical dosages range between about 5 mg and about 60 mg. Narcotics such as codeine, dihydrocodeine, hydrocodone, hydromorphone, oxycodone, pentazocine propoxyphene, and the like are also suitable for use as analgesics. Alkaloids such as dihydroergotamine and ergotamine are also useful as analgesics.

Exemplary skeletal muscle relaxants that can be incorporated in the delivery systems include, but are not limited to, carisoprodol, chlorphenesin, chlorzoxazone, metaxalone and methocarbamol. Exemplary antacids include, but are not limited to, calcium carbonate, alumina and magnesium oxide.

Exemplary anticholinergics include, but are not limited to, atropine, hyoscyamine, methscopolamine and scopolamine. Typical dosages range between about 300 μg and about 60 mg.

As is known in the art, certain functional ingredients can act to enhance in the effect of NSAIDs in the body. Such “potentiators” can also be included in the delivery systems. Examples of suitable potentiators include, but are not limited to, B vitamins, dextromethorphan, diphenylhydramine and caffeine.

Phospholipids, in the form of lamellar bodies, have also been reported to enhance or potentiate the analgesic and anti-inflammatory effects of NSAIDS in a human or other mammal (see, International Patent Application WO 97/268890) and can also be included in the delivery systems.

As indicated above, typically the total amount of NSAID(s) and other functional ingredients constitute up to about 40% by weight of the delivery system. Thus, the amount of other functional ingredient(s) included in the delivery system will be dependent on the total amount of NSAID(s) that is to be incorporated. As indicated above, based on the average effective dosage of NSAID(s), the NSAID(s) may constitute less than about 10% by weight of the delivery system. Accordingly, in one embodiment of the present invention, the delivery systems incorporate between about 0.01% and about 30% by weight of other functional ingredient(s) in addition to the one or more NSAID. In another embodiment, the delivery systems incorporate between about 0.01% and about 25% by weight of other functional ingredient(s) in addition to the one or more NSAID. In another embodiment, the delivery systems incorporate between about 0.01% and about 20% by weight of other functional ingredient(s). In another embodiment, the delivery systems incorporate between about 0.01% and about 15% by weight of other functional ingredient(s). In another embodiment, the delivery systems incorporate between about 0.01% and about 10% by weight of other functional ingredient(s). In a further embodiment, the delivery systems incorporate between about 0.01% and about 5% by weight of other functional ingredient(s).

4. Bioavailability Enhancers

The present invention also contemplates the inclusion of bioavailability enhancers in the delivery systems. Such compounds are known in the art and act to increase the absorption of functional ingredients by the body. Bioavailability enhancers can be natural or synthetic compounds.

Natural bioavailability enhancers include ginger, caraway extracts, pepper extracts and chitosan. The active compounds in ginger include 6-gingerol and 6-shogoal. Caraway oil can also be used as a bioavailability enhancer (U.S. Patent Application 2003/022838). Piperine is a compound derived from pepper (Piper nigrum or Piper longum) that acts as a bioavailability enhancer (see U.S. Pat. No. 5,744,161). Piperine is available commercially under the brand name Bioperine® (Sabinsa Corp., Piscataway, N.J.). Natural bioavailability enhancers can be present in an amount of from 0.02% to 0.6% by weight based on the total weight of the delivery system.

Synthetic bioavailability enhancers are typically based on macrogol glycols and glycerides or polyethylene glycol (PEG). Examples of suitable synthetic bioavailability enhancers include, but are not limited to, Gelucire®, Labrafil® and Labrasol®, Lauroglycol®, Pleurol Oleique®, (Gattefossè Corp., Paramus, N.J.) and Capmul® (Abitec Corp., Columbus, Ohio).

The amount of synthetic bioavailability enhancer that can be included in the delivery systems is typically defined by the ratio of synthetic bioavailability enhancer to NSAID(s). This ratio can vary between about 1.0:10.0 and 10.0:1.0. In one embodiment of the present invention, the synthetic bioavailability enhancer to NSAID(s) ratio varies between about 1.0:10.0 and 5.0:1.0. In another embodiment of the present invention, the synthetic bioavailability enhancer to NSAID(s) ratio varies between about 1.0.10.0 and 3.0:1.0

One or more of the above-described bioavailability enhancers may be included in the delivery systems in order to enhance the bioavailability of the NSAID(s) and/or other functional ingredients.

### Process For Preparing The Delivery System

In accordance with the present invention, the delivery system remains flowable at temperatures below 100° C. which allows for fall dispersion and incorporation of the NSAID(s) and other optional functional ingredients into the matrix while minimising or preventing degradation of these compounds. Thus, although the actual methodology used to prepare the delivery systems may vary depending on the individual components selected to make up the matrix, the process of preparing the matrix comprises the step of incorporating the NSAID(s) and other optional functional ingredient(s) into the matrix at temperatures below 100° C. In one embodiment of the present invention, the process of preparing the matrix comprises the step of incorporating the functional ingredient(s) into the matrix at temperatures below about 75° C. In another embodiment, the process of preparing the matrix comprises the step of incorporating the functional ingredient(s) into the matrix at temperatures below about 65° C. In another embodiment, at least one functional ingredient is dispersed in the solvent component prior to admixture with the other matrix components.

Various standard methods known in the confectionery manufacturing industry can be used to prepare the delivery systems and selection of the appropriate method is considered to be within the ordinary skills of a worker in the art. Batch processes, such as kettle cooking, as well as continuous processes, such as direct steam injection jet cookers and indirect steam tubular heat exchangers, are suitable for preparing the delivery system.

The following description represents a general method of preparing a delivery system of the present invention.

Briefly, the process comprises the following steps: a blend of the hydrocolloid component and the sugar component, and optionally water, is prepared. A ratio of components is selected that will result in a final product with the desired moisture content (i.e. 10%-40%). The hydrocolloid(s) may be pre-hydrated in water or may be hydrated during this blending step. The blend is heated to a temperature of less than 100° C., for example between 60° C. and 80° C., such that all ingredients are incorporated. Alternatively, the sugar component, and optionally water, can be heated to a temperature of less than 100° C. (for example between 60° C. and 80° C.) prior to addition of the dry or pre-hydrated hydrocolloid(s) under shear. The temperature of the mixture is then reduced to between 50° C. and 80° C. The NSAID(s) and/or other optional functional ingredient(s) are dispersed or dissolved in solvent at or below 70° C., for example below 50° C. If required, one or more sources of mono- or divalent cations and one or more pH adjusting agents can be added to either, or both, of the above preparations. The two preparations are then combined. Flavourings and colourings may optionally be added after this step.

As an alternative to adding pH adjusting agents as indicated above, the pH of the matrix can be adjusted, as necessary, after combining the two preparations. Suitable methods of adjusting the pH of food products are known in the art and include, for example, the addition of buffers, acids or bases, such as citric acid, sodium citrate, phosphates, sodium hydroxide, potassium hydroxide or a combination thereof.

As indicated above, the final product has a moisture level between 10% and 40%, for example between 15% and 20%, and a water activity of less than 0.9.

In one embodiment of the invention, the process includes the step of heating the blend of hydrocolloid(s) and the sugar component (and optionally water) to a temperature between about 60° C. and about 70° C. In another embodiment, the process includes the step of heating the sugar component, and optionally water, to a temperature between about 60° C. and about 70° C. prior to addition, under shear, of the dry or pre-hydrated hydrocolloid(s). In a further embodiment, the process includes the step of dispersing or dissolving the NSAID(s) and/or other optional functional ingredient(s) in the solvent at a temperature between about 40° C. and about 50° C.

Once the matrix has been prepared as described above, it can then be moulded, for example, using the standard Mogul process or by injection-filling of pre-formed moulds. One skilled in the art will appreciate that the matrix can also be readily adapted to extrusion methods.

In final form, the delivery systems of the present invention are semi-solid, intermediate moisture systems, having some properties clearly identified with those of jellies and some properties that are similar to the jujube variety of confectioneries. The matrix of the delivery systems is thus formulated to be semi-solid at normal room temperature (i.e. at temperatures between about 20° C. and about 30° C.). It will be readily apparent that depending on the particular components selected for use in the preparation of the matrix, the amount of each to be included in the matrix may need to be manipulated within the ranges indicated in order to achieve a semi-solid, intermediate moisture product. One skilled in the art of confectionery design can readily determine which component(s) will need to be adjusted in order to achieve an end-product with these physical properties.

Similarly, it will be readily apparent to one skilled in the art that variations can be made to the described process dependent on the type and the actual amount of each component used (within the given ranges) in order to obtain an end product with the described properties. For example, if the hydrocolloid comprises a starch, it is known in the art that the gelatinisation temperature of the starch may be affected when certain sugars and sugar alcohols are used. If required, therefore, the starch and the sugar component can be heated above 100° C. to allow fall gelatinisation of the starch to occur and the desired moisture content to be reached. The temperature of the mixture can then be reduced to between 50° C. and 80° C. prior to addition of the functional ingredient(s) and optionally flavourings and colourings.

As is known in the art, modified celluloses, such as methylcellulose and hydroxypropyl methylcellulose, have unique properties resulting in the ability to delay hydration of these carbohydrates during preparation processes. Thus, when these compounds are used a “delayed hydration technique” may be employed in which the modified cellulose is first dispersed in the solvent component of the matrix and then mixed with the other components in aqueous solution. The hydration of the modified cellulose then takes place gradually as the processing is complete and the moulded matrix cools. Delayed hydration and non-aqueous fluid carrier techniques using modified celluloses are standard in the art.

Similarly, the choice of hydrocolloid can affect the set up temperature of the matrix. The use of a combination of starch, gelatine and gellan, for example, can provide a matrix set-up temperature of about 35° C., as can a combination of starch, gelatine and pectin. In contrast, the use of other hydrocolloids or combinations of other hydrocolloids with or without gelatine or gellan, may alter the set up temperature of the matrix. For example, the use of starch in combination with locust bean gum or carageenan often results in set up temperatures of around 60° C. The choice of hydrocolloid is thus dependent on the functional ingredient(s) to be incorporated into the matrix. Temperature sensitive functional ingredients will require a hydrocolloid or hydrocolloid mixture that provides a low set up temperature (such as the gelatine:gellan or gelatine:pectin mixtures described above), whereas other hydrocolloids or mixtures thereof can be used with functional ingredients that can tolerate higher temperatures.

The manner in which the individual components are combined may also be varied although typically at least one of the functional ingredients is dispersed in solvent prior to addition to the remainder of the components. For example, the sugar component may be heated with the water and salts prior to addition of the hydrocolloid(s). Similarly, when two or more hydrocolloids are being used, they do not have to be added to the mixture at the same time. One hydrocolloid and part of the sugar component could be mixed and heated prior to being blended with the other hydrocolloid and remainder of the sugar component. Alternatively, one hydrocolloid and the sugar component could be mixed and heated prior to addition of the second hydrated hydrocolloid, or one hydrocolloid may be added to the solvent component and then blended with the second hydrocolloid and sugar component. These and other variations are considered to be within the scope of the present invention.

Testing The Delivery System

1. Physical Properties

One skilled in the art will appreciate that molecular interaction between one or more of the functional ingredients and the matrix may affect the physical attributes of the final product. As is standard in the art, therefore, a sample of the delivery system incorporating the NSAID(s) and optionally other functional ingredient(s) can be prepared prior to large-scale production and tested in order to determine whether the matrix retains the desired physical properties, i.e. substantially uniform dispersion of the NSAID(s) and other functional ingredients, less than 20% degradation of these compounds during the preparation of the matrix and water activity less than 0.9.

For example, dispersion of the NSAID(s) in the final delivery system can be determined by dividing a single unit of the delivery system into several subunits and analysing the content of NSAID in each subunit, for example as a % by weight. The levels of NSAID can readily be measured by standard analytical techniques such as mass spectrometry, UV or IR spectrometry, or chromatographic techniques, such as gas chromatography or high-performance liquid chromatography (HPLC). If the % by weight of NSAID in each subunit is similar, then the NSAID is said to be substantially uniformly dispersed throughout the product. One skilled in the art will appreciate that the % by weight need not be identical for each subunit to indicate substantially uniform dispersion. In accordance with the present invention, the % by weight of NSAID for each subunit of the final delivery system varies by less than 2%. In one embodiment, the % by weight of NSAID for each subunit of the final delivery system varies by less than 1.5%. In other embodiments, the % by weight of NSAID for each subunit varies by less than 1% and by less than 0.5%.

The dispersion of other functional ingredients incorporated into the delivery system can also be measured as described above.

Similarly, the degradation of the functional ingredient(s) can be determined by standard analytical techniques taking into account the total amount of each compound included in the preparation of the matrix. Many functional ingredients degrade to yield specific breakdown products, the presence or absence of which can be determined in the final product using standard techniques, such as spectrophotometric and chromatographic techniques, e.g. gas chromatography and HPLC. As indicated above, the degradation of the functional ingredients is minimised during the preparation of the delivery system and is less than about 20% in the final product.

The water activity (a_{w}) of the final product can also be analysed by standard techniques. The a_{w} of a food product is a physical property that has direct implications on the microbial safety of the product and influences storage stability. Lower a_{w} values generally indicate a food product that is more stable and more resistant to microbial contamination than one with a high a_{w} value due to the requirement for water of most microbes and the fact that most deteriorative processes in food products are mediated by water. As is known in the art, the a_{w} value of a food product is the ratio of the water vapour pressure of the product (p) to that of pure water (pₒ) at the same temperature, i.e. a_{w}=p/pₒ. In accordance with the present invention, the water activity of the final delivery system is less than about 0.9, for example between about 0.5 and about 0.7.

Other parameters, such as the release rate of the functional ingredients from a delivery system can also be tested by standard methods (for example, the USP Basket Method or Paddle Method; see U.S. Pharmacopoeia XXII (1990)). Typically, a sample of the delivery system containing a known amount of functional ingredient(s) (for example, a unit dose) is placed in an aqueous solution of a predetermined pH, for example around pH 1.2 to simulate stomach conditions and/or around pH 7.4 to simulate colon conditions. The suspension may or may not be stirred. Samples of the aqueous solution are removed at predetermined time intervals and are assayed for their content of the NSAID(s) and other optional functional ingredients by standard analytical techniques, such as those indicated above.

In addition, the delivery system may undergo testing to evaluate such factors as the microbial content of the product and the shelf-life of the product. Such quality control testing is standard in the art and can be conducted using known methods.

For example, microbial analysis of the delivery system can be conducted using techniques approved by the appropriate regulatory board, such as those described in “The Compendium of Analytical Methods: HPB Methods for the Microbiological Analysis of Foods” issued by the Health Products and Food Branch of Health Canada. Shelf life is typically evaluated using accelerated shelf life tests in which the stability of the system and the degradation of the functional ingredients contained therein is analysed under conditions that are known to accelerate the degradation of food products and can be correlated to the stability of the product under normal storage conditions.

Texture measurements can also be made to determine whether the delivery system has the required gel strength/hardness. Gel strength or hardness can be measured either directly (expressed as grams force) and indirectly (expressed as a viscosity), or both.

Methods of measuring gel hardness are known in the art. For example, a Kramer single blade shear cell can be used. In this test, a shear blade is driven down at a constant speed through a sample of the delivery system and the peak force as the blade cuts through the sample is measured. The test force is typically reported in kilograms-force. Various machines are available to conduct such testing, for example, a Universal Testing machine such as that available from Instron or Stable Micro Systems (e.g. the Model TA.HD Texture Analyzer).

Gel hardness can also be measured using a standard Brookfield viscometer (e.g. the Model RVDV), which measures the force required to cut through a gelled liquid. A spindle rotating at a set speed is slowly lowered into a sample of the delivery system and the torque required for the spindle to “cut” through the sample is measured. Temperature is important to obtain an accurate viscosity reading and thus the samples are usually tempered to 21° C. to 24° C. prior to testing. The cutting force or torque reading on the viscometer is an empirical measure of gel strength and is reported in centipoise (cps).

Another method useful for measuring sensory texture utilises the Hamann Torsion/Vane Gelometer. This system provides fracture shear stress and shear strain values and real time test graphs of stress vs. strain or angular deformation. Stress (strength) and strain (deformability) are not “geometrically coupled” as in most traditional (empirical) textural tests, therefore, the strain measurement remains unaffected by the magnitude of the stress measurement. Strain has been found to be the best indicator of gelling quality for proteins and hydrocolloids, as this parameter is less sensitive to concentration effects, and is also a good indicator of the perceived “rubberiness” of food gels. Strain values also predict machining characteristics of food gels, such as ease of slicing. Furthermore, the sample shape does not change during testing with the Torsion Gelometer, thus minimal fluids will be forced from the sample during testing and the gel itself is tested rather than a dehydrated derivative. The mode of failure in torsion testing yields important information about the texture of the sample. Test samples of the delivery system are formed in either cylindrical molds (tubes) for subsequent milling, which eliminates surface skin effects, or in a dumbbell mold. Samples are then cut to a standard length (for example, 1 inch) and loaded into the measuring cell for testing. Data collection continues for a time past the breaking of the sample (peak stress or Fracture Point). Stress (in kPa), strain, rigidity modulus (G=stress/strain) and slope ratio at failure can be measured in this method

Palatability can also be tested using standard techniques. Methods of evaluating the organoleptic properties of foods are well-known in the art. For example, sensory evaluations can be performed using individuals who are spatially separated from each other, for example, in individual partitioned booths, as testers and a hedonic nine-point scale that ranges from 1 (most disliked) to 9 (most liked), with 5 indicating no preference [Larmond, Laboratory methods for Sensory Evaluation of Foods, Research Branch of Agriculture Canada (1977)]. Odour and taste are generally evaluated under a red light, which masks any differences in the colour of the product. Another nine-point hedonic scale test can be carried out under normal light to evaluate the acceptability of the appearance of the product.

2. Efficacy

The delivery systems of the present invention can be tested for efficacy in vivo. Typically, the efficacy is tested by conducting bioavailability studies using standard techniques in the pharmaceutical art, such as peak plasma levels and pharmokinetic analyses (see, for example, Enna, et al., Current Protocols in Pharmacology, J. Wiley & Sons, New York, N.Y.).

Bioavailability studies are usually conducted by administering to groups of subjects various doses of the delivery system under study over a pre-determined period of time and comparing plasma levels of NSAID in these groups at varying intervals with an appropriate control or controls. Appropriate controls include groups of subjects taking recommended doses of competitor's products (i.e. positive controls) and groups of subjects taking a placebo or no drug (i.e. negative controls). The subjects may or may not have fasted prior to administration of the doses of the delivery system. Single dose or multiple dose studies may be conducted. The studies can also be used to monitor any side-effects of the dosing regimens of the delivery system under investigation by compiling reports of any adverse effects encountered during the course of the study and comparing them to side-effects reported by the control group(s). Optimal dosing schedules can also be determined in this manner.

Studies to determine that the combination of functional ingredients in a delivery system bring about the desired effect, for example alleviation of pain or a decrease in inflammation and/or fever, in a subject can also be conducted in a similar manner to the bioavailability studies indicated above. Such studies are routine in the art and can be readily designed and conducted by a skilled technician.

Format Of The Delivery System

The present invention contemplates various formats for the delivery systems. For example, the delivery systems may be in the form of a confectionery, such as a jujube, in which case it may be formulated alone or it may further comprise a coating, such as a chocolate or yoghurt coating. Preparation of jujube or jelly type confectionery products are known in the art and include, for example, the use of moulds, injection-filling of pre-formed packages and extrusion processes. It will be readily apparent to one skilled in the art that such standard techniques can be applied to prepare a wide variety of different shaped confectioneries.

The present invention further contemplates the delivery system as a filling or a coating, for example, for baked goods such as wafers or cookies. For example, the matrix can be used as a layer between two wafers, or a jelly layer on the top of a cookie or sponge, in which case the product may be further coated with a chocolate or other flavoured coating, if desired, as described above for confectionery products. Alternatively, the matrix may be used to fill doughnut type baked goods. Methods of filling and coating baked goods are also well known in the art.

Administration And Use

The one or more selected NSAIDs and the other optional functional ingredients are incorporated into the delivery systems of the invention at levels sufficient to bring about the desired analgesic, antipyretic, anti-inflammatory and/or prophylactic effect in the body when taken regularly. The exact amount of NSAID to be included in a particular delivery system will be dependent, for example, on the specific NSAID(s) being utilised, the condition for which the drug is being administered and the size and type of animal being treated.

Typical unit doses for NSAIDs are known in the art (see, for example, Physician's Desk Reference, 57^{th} Edition, 2003). Representative oral doses for an adult human of some common NSAIDs are provided in Table 1.

TABLE 1
Adult Human Dose Ranges for Common NSAIDs

| NSAID | Dose Range | |
|---|---|---|
| Acetaminophen | 150 to 650 | mg |
| Aspirin | 81 to 770 | mg |
| Diclofenac | 25 to 100 | mg |
| Indomethacin | 25 to 75 | mg |
| Ibuprofen | 100 to 200 | mg |
| Ketoprofen | 25 to 200 | mg |
| Celecoxib | 100 to 400 | mg |

The delivery systems of the present invention can be administered to a patient in order to relieve pain, which may be chronic or acute, to reduce inflammation and/or to reduce fever. Alternatively, as described above, the delivery systems can be employed for prophylactic purposes with the aim of minimising inflammation and/or pain that may occur as the result of imminent surgery, strenuous exercise, or the like. The delivery systems are thus useful in a variety of situations, for example, for the relief of pain and/or inflammation associated with arthritis (including osteoarthritis, rheumatoid arthritis, juvenile arthritis, ankylosing spondylitis and gout), rheumatism, soft tissue trauma, sport's injuries, migraines, tension headaches, dysmenorrhoea, surgical procedures, tendinitis, bursitis, as well as for relief of dental pain, oral pain, musculoskeletal pain, joint pain, and the like. The delivery systems can also be used to alleviate symptoms associated with colds, influenza and other viral or bacterial infections.

The present invention further contemplates that the delivery systems may be formulated to comprise low doses of acetyl salicylic acid, or a derivative thereof, which are useful for blood thinning applications, such as prevention of blood clot formation. Low-dose acetyl salicylic acid delivery systems can thus be used to lessen the chance of heart attack, stroke, or other problems that may occur when a blood vessel is blocked by blood clots. For this purpose, doses of 80 to 1000 mg a day are useful, more typically between 80 to 325 mg a day.

Specific combinations of functional ingredients can be included in the delivery systems in order to provide relief from a particular set of symptoms in a patient. For example, delivery systems can be formulated for the treatment of a patient suffering from a cold or influenza that comprise a combination of one or more NSAID with one or more of: an anti-tussive, anti-histamine, expectorant, non-NSAID analgesic, anticholinergic and decongestant.

Delivery systems comprising a combination of one or more NSAID with one or more of: an anti-histamine, decongestant and anticholinergic can be used for relieving symptoms due to allergies and hay fever, as well as the common cold. Caffeine can be used in combination with the antihistamine to overcome the drowsiness caused by the antihistamine.

Delivery systems comprising a combination of one or more NSAID with one or more other anti-inflammatory can be useful in treating pain associated with rheumatism, arthritis, infections and other conditions in which inflammation occurs.

Combining one or more NSAID with an antacid in a delivery system can help to minimise the undesirable gastric side-effects that are associated with some NSAIDs.

For severe pain relief a delivery system can be formulated that comprises one or more NSAIDs in combination with a narcotic analgesic and,optionally a CNS stimulant. For example, caffeine in combination with ergotamine and one or more NSAID is useful in the treatment of migraine and cluster headaches.

NSAIDs can also be combined with B vitamins for the treatment of moderate to severe pain. For example, diclofenac co-administered with B vitamins has been demonstrated to be more effective in relieving pain than diclofenac alone [see, Vetter G. et al, Z Rheumatol. (1988) 47:351-62].

Delivery systems can be formulated for the treatment of a patient suffering from tension headaches that comprise a combination of one or more NSAIDs with one or more muscle relaxant.

Combinations of one or more NSAIDs with one or more anticholinergics/antispasmodics can be used to formulate delivery systems for the relief of cramps or spasms of the stomach, intestines, and bladder, for relief of pain associated with dysmennorhoea and to help prevent nausea, vomiting, and motion sickness.

Delivery systems comprising one or more NSAIDs, one or more anticholinergics/antispasmodics and an antacid can be useful in the treatment of peptic ulcer.

It will be readily apparent that the delivery systems of the invention are suitable for use by a variety of individuals who are in need of relief from pain, inflammation, fever and the like, as well as individuals who may potentially be at risk of developing inflammation or experiencing minor or severe pain and/or aches.

In addition to the uses outlined above, the delivery systems of the present invention find application in the realm of sports nutrition. Sports nutrition is associated with the intake of functional ingredients that affect various factors relating to an individual's endurance, performance, recovery, energy levels, weight maintenance, and the like. The NSAID delivery systems of the present invention are thus useful in alleviating pain and/or inflammation associated with sport's injuries or associated with conditions that may otherwise impact an individual's ability to perform optimally. The present invention farther contemplates delivery systems comprising a combination of one or more NSAIDs with other functional ingredients intended to increase endurance, improve performance and/or reduce recovery time. The prophylactic applications of NSAIDs can be important in this regard. By way of example, delivery systems can be designed that comprise one or more NSAIDs in combination with one or more ergogenic compounds, such as amino acids and their salts, antacids, antihistamines, antioxidants, bee pollen, beta-blockers, benzodiazapines, β₂-agonists, bicarbonates, caffeine, carbohydrates, carnitine, choline, coenzyme Q10, creatine, DHEA, ephedra, folic acid, ginseng, guarana, calcium beta-hydroxy beta-methylbutyrate, inosine, minerals (such as boron, calcium, chromium, iron, magnesium, selenium, zinc), niacin, phosphates, protein, pyruvate and vitamins B₁, B₂, B₆, B₁₂ C, E, where such combinations are not contraindicated.

The delivery systems of the invention can be formulated in various unit sizes depending on the amount of NSAID(s) and other functional ingredients to be incorporated therein and on requirements of the target consumer. The delivery systems of the present invention can be formulated to have a unit size between about 3 grams and about 30 grams. In one embodiment, a unit of the delivery system is between about 3 grams and about 20 grams. In another embodiment, a unit of the delivery system is between about 3 grams and about 15 grams. In another embodiment, a unit of the delivery system is between about 3 grams and about 10 grams. Where appropriate, the delivery systems can be provided in a multi-dose format that is pre-scored into unit doses.

The delivery systems can be formulated for administration to humans or other animals. For administration to humans, flavours and formats that appeal to the particular group of consumers being targeted can be employed. For example, delivery systems that are formulated with confectionery-like qualities and flavours are appealing to children who are often resistant to taking medications or supplements due to unpleasant tastes or mouthfeel.

Similarly, the delivery systems can be formulated for administration to a non-human animal using flavours that more typically appeal to non-human animals, for example, fish, poultry or meat flavours. Administration of functional ingredients to an animal in conventional solid dosage forms, such as tablets and capsules, can be problematic in that the animal often expels them, and multiple dosing is often difficult because the animal learns to resist the dosing procedure. It will be readily apparent that the delivery system of the present invention, which is formulated as a foodstuff, is ideally suited for administration of NSAID(s) to animals.

Kits

The present invention additionally provides for kits containing a NSAID delivery system for administration to a human or non-human animal. The kit would provide an appropriate dosing regimen over a prescribed period for the NSAID(s) and other functional ingredient(s) contained in the delivery system.

The kits of the invention comprise one or more packages containing the delivery system and may further comprise a set of instructions, generally written instructions, relating to the use and dosage of the NSAID(s) and other optional functional ingredient(s) contained in the delivery system. The instructions typically include information as to the appropriate dosage and dosing schedule for the functional ingredients in terms of units of the delivery system. The packages containing the delivery system may in the form of unit doses, bulk packages (for example, multi-dose packages) or sub-unit doses. The doses may be packaged in a format such that each dose is associated, for example, with a day of the week. There may also be associated with the kit a notice in the form prescribed by a governmental agency regulating the manufacture, use or sale of biological products, which notice reflects approval by the agency of manufacture, use or sale for human or animal administration.

To gain a better understanding of the invention described herein, the following examples are set forth. It should be understood that these examples are for illustrative purposes only. Therefore, they should not limit the scope of this invention in any way. All percentages throughout the specification and claims are by weight of the final delivery system unless otherwise indicated.

### EXAMPLES

### Example 1

NSAID Delivery Systems

The delivery systems described below are formulated to have a final pH between 5.0 and 9.0, more typically between 6.5 and 8.5. The delivery systems have a final a_{w} between about 0.5 and about 0.6.

1.1 Delivery System for Ibuprofen

The following delivery system was formulated to deliver 200 mg of ibuprofen in a 13 g product. The moisture content of the final delivery system was approximately 16.9% by weight.

| Ingredient | % by Weight |
|---|---|
| Glycerol | 35.56% |
| Propylene glycol | 2.09% |
| Ibuprofen | 1.54% |
| 63 DE Corn syrup | 19.24% |
| High Fructose Corn Syrup | 22.59% |
| Gelatine | 8.58% |
| Pectin | 0.31% |
| Sweetening agents | 0.12% |
| Modified Starch | 1.92% |
| Flavour | 0.18% |
| Colour | 0.35% |
| Water | 7.53% |
| Total: | 100.01% |

1.2 Delivery System for Acetaminophen

The following delivery system was formulated to deliver 200 mg of acetaminophen in an 11.5 g product. The moisture content of final delivery system was approximately 16.9% by weight.

| Ingredient | % by Weight |
|---|---|
| Glycerol | 35.48% |
| Propylene glycol | 2.09% |
| Acetaminophen | 1.74% |
| 63 DE Corn syrup | 19.20% |
| High Fructose Corn Syrup | 22.54% |
| Gelatine | 8.58% |
| Pectin | 0.30% |
| Sweetening agents | 0.12% |
| Modified Starch | 1.92% |
| Flavour | 0.18% |
| Colour | 0.35% |
| Water | 7.51% |
| Total: | 100.01% |

1.3 Delivery System for Diclofenac

The following delivery system was formulated to deliver 50 mg Diclofenac in an 11 g final product.

| Ingredient | % by Weight |
|---|---|
| Glycerol | 35.83% |
| Propylene glycol | 2.11% |
| Diclofenac sodium | 0.46% |
| 63 DE Corn syrup | 19.39% |
| High Fructose Corn Syrup | 22.76% |
| Gelatine | 8.64% |
| Pectin | 0.31% |
| Sweetening agents | 0.12% |
| KOH | 0.32% |
| Modified Starch | 1.94% |
| Flavour | 0.18% |
| Colour | 0.36% |
| Water | 7.59% |
| Total: | 100.00% |

1.4 Delivery System for Indomethacin

The following delivery system was formulated to deliver 25 mg Indomethacin in a 6 g final product.

| Ingredient | % by Weight |
|---|---|
| Glycerol | 35.96% |
| Propylene glycol | 2.11% |
| Indomethacin | 0.42% |
| 63 DE Corn syrup | 19.29% |
| High Fructose Corn Syrup | 22.67% |
| Gelatine | 8.64% |
| Pectin | 0.31% |
| Sweetening agents | 0.12% |
| KOH | 0.42% |
| Modified Starch | 1.94% |
| Flavour | 0.18% |
| Colour | 0.36% |
| Water | 7.59% |
| Total: | 100.00% |

1.5 Delivery System for Ibuprofen with a Bioavailability Enhancer

The following delivery system was formulated to deliver about 100 mg Ibuprofen in a 3 g final product.

| Ingredient | % by Weight |
|---|---|
| Glycerol | 30.19% |
| Propylene glycol | 2.09% |
| Ibuprofen | 3.33% |
| Gelucire 44/14 | 3.33% |
| 63 DE Corn syrup | 19.24% |
| High Fructose Corn Syrup | 22.56% |
| Gelatine | 8.58% |
| Pectin | 0.31% |
| KOH | 0.26% |
| Sweetening agents | 0.12% |
| Modified Starch | 1.92% |
| Flavour | 0.18% |
| Colour | 0.35% |
| Water | 7.53% |
| Total: | 100.00% |

The above NSAID formulations (1.1 to 1.5) were prepared by the following general method:

The glycerol and propylene glycol were blended and the NSAID dispersed therein and the blend warned to 40-55° C. The sugar syrups were blended with the water and warmed to 60-70° C. The gelatine, pectin, sweetening agents and other dry ingredients were preblended and introduced into the syrup under shear. The NSAID blend was then uniformly blended with the gelatine preparation. Flavour and colour were then added and the whole maintained between 40° C. and 55° C.

### Example 2

Delivery Systems using Other Functional Ingredients

The following delivery systems (formulated using functional ingredients other than NSAIDs) demonstrate how the components of the matrix can be varied. These systems can be readily adapted for NSAID delivery by a worker skilled in the art, by replacing the listed functional ingredients with one or more NSAID and optionally, one or more other functional ingredient, in accordance with the present invention. A worker skilled in the art will recognise that the ingredients in the following formulations may need to be adjusted proportionally when adapting the formulations to deliver small amounts of NSAID. In addition, the use of pH modifying or buffering ingredients included when formulating with specific functional ingredients may not be required when adapting the formulations to deliver a NSAID. The moisture content of the following delivery systems was between about 13% and about 17% by weight.

2.1

| Ingredient | % by Weight |
|---|---|
| Glycerol | 14.57% |
| Propylene Glycol | 5.30% |
| Functional ingredients* | 13.38% |
| Corn Syrup 62DE | 31.79% |
| Sucralose | 0.04% |
| Modified Starch | 2.65% |
| Potassium citrate | 2.15% |
| High fructose corn syrup | 9.27% |
| Water | 14.57% |
| Gelatine 100 bloom type B | 1.32% |
| Gelatine 250 bloom type A | 3.97% |
| Gellan (Kelcogel ® LT100) CP Kelco | 0.32% |
| Colour | 0.21% |
| Flavour | 0.45% |
| Total: | 100.00% |

*creatine monohydrate (11.71%) and dimethylglycine (1.67%)

2.2

| Ingredient | % by Weight |
|---|---|
| Glycerol | 12.57% |
| Propylene Glycol | 4.19% |
| Functional ingredient (arginine) | 14.02% |
| Maltitol solution | 33.52% |
| Modified Starch | 2.79% |
| Potassium citrate | 1.17% |
| Sucralose | 0.04% |
| High fructose corn syrup | 9.78% |
| Water | 15.37% |
| Gelatine 250 bloom type A | 5.59% |
| Gellan (Kelcogel ® LT100) CP Kelco | 0.28% |
| Colour | 0.168% |
| Flavour | 0.503% |
| Total: | 100.00% |

2.3

| Ingredient | % by Weight |
|---|---|
| Glycerol | 13.82% |
| Propylene Glycol | 5.53% |
| Functional ingredients* | 11.02% |
| Isomalt syrup | 33.17% |
| Sucralose | 0.055% |
| Modified Starch | 2.76% |
| Potassium citrate | 2.24% |
| High Fructose Corn syrup | 9.68% |
| Water | 15.20% |
| Gelatine 250 bloom type A | 5.53% |
| Gellan (Kelcogel ® LT100) CP | 0.33% |
| Colour | 0.08% |
| Flavour | 0.08% |
| Total: | 100.00% |

*creatine monohydrate (4.59%), conjugated linoleic acid (CLA; 4.59%), lecithin (1.05%), N,N,dimethylglycine (0.47%), rhodiola/seabuckthorn extract solution (0.21%) and chromium chelate (0.11%).

2.4

| Ingredient | % by Weight |
|---|---|
| Glycerol | 14.82% |
| Propylene Glycol | 5.39% |
| Functional ingredient (creatine monohydrate) | 11.91% |
| Corn Syrup 62DE | 32.33% |
| Sucralose | 0.04% |
| Modified Starch | 2.70% |
| Potassium citrate | 2.19% |
| High fructose corn syrup | 9.43% |
| Water | 14.82% |
| Gelatine 100 bloom type B | 1.34% |
| Gelatine 250 bloom type A | 4.04% |
| Gellan (Kelcogel ® LT100) CP Kelco | 0.33% |
| Colour | 0.21% |
| Flavour | 0.46% |
| Total: | 100.00% |

The above formulations were prepared by the following general method:

Glycerol and propylene glycol were first blended and at least one functional ingredient was added. The blend was heated to 65-70° C. In a separate container, gelatine and gellan were blended together. The fructose syrup and water were mixed and heated to 60° C., after which the gelatine:gellan mixture was added with constant agitation. The mixture was then heated to 75° C. to allow the components to dissolve. In a third container, the syrup was warmed to 30-35° C. and the sucralose, potassium citrate, other functional ingredients and starch were then blended in. The syrup mixture was combined with the gelatine:gellan mixture and heated to 75-80° C. until the moisture content was reduced and the desired solids level achieved. The glycerol mixture was then added together with the colour and flavour additives. The delivery system was then moulded using standard techniques.

2.5

| Ingredient | % by Weight |
|---|---|
| Glycerol | 27.9990% |
| Propylene Glycol | 3.4145% |
| Potassium Hydroxide | 0.1208% |
| Functional ingredient (creatine monohydrate) | 24.0154% |
| High Fructose Corn Syrup | 15.7068% |
| Corn syrup | 14.7962% |
| Modified Starch | 2.5040% |
| Water | 3.9836% |
| Potassium phosphate | 0.4234% |
| Sucralose | 0.0381% |
| Potassium citrate | 0.9526% |
| Gelatine Type A | 4.7803% |
| Pectin | 0.2732% |
| Flavour | 0.5464% |
| Colour | 0.2982% |
| Total: | 100.0000% |

The following method was used to prepare the above delivery system. Glycerol and propylene glycol were first blended and the creatine was added. The blend was heated to 45-50° C. In a separate container, the gelatine, pectin, starch and sucralose were blended together. The fructose and glucose syrups and water were mixed and heated to 60° C., after which the salts and pH modifying agents were added with constant agitation and heated to 60-70° C. to dissolve the solids. The powder blend was then incorporated into the syrup mixture using high shear. Finally, the creatine mixture was added, together with the colour and flavour additives, and blended. The delivery system was then moulded using standard techniques.

2.6

| Ingredient | % by Weight |
|---|---|
| Glycerol | 16.67% |
| Propylene Glycol | 7.86% |
| Functional ingredients* | 9.36% |
| Maltitol syrup | 35.86% |
| High fructose corn syrup | 15.73% |
| Sucralose | 0.06% |
| Modified Starch | 3.15% |
| Potassium citrate | 1.42% |
| Potassium hydroxide | 0.92% |
| Water | 1.38% |
| Gelatine | 6.29% |
| Pectin | 0.31% |
| Colour | 0.3% |
| Flavour | 0.74% |
| Total: | 100.00% |

*Conjugated linoleic acid (Clarinol 80; 7.86%), citrus aurantium (0.5%), inulin (0.63%), caffeine (0.25%), mixed tocopherols (0.04%) and ascorbic acid (0.03%).

The following method was used to prepare the above delivery system. The glycerol and propylene glycol were first blended together. At least one functional ingredient was then added and the resultant mixture was warmed to 60-70° C. In another container, the syrups, water, potassium citrate and potassium hydroxide were combined and warmed to 60-70° C. The starch, gelatine, pectin, sucralose and remaining functional ingredients were pre-blended then added to the syrup mixture under high shear. This mixture was combined with the glycerol mixture and the temperature maintained at 60-70° C. until the moisture content was reduced sufficiently to give the desired solids level. Colour and flavour were added and the mixture was then moulded using standard techniques.

2.7

| Ingredient | % by Weight |
|---|---|
| Glycerol | 15.97% |
| Propylene Glycol | 5.51% |
| Functional ingredient (creatine monohydrate) | 16.71% |
| 63 DE Corn syrup | 21.20% |
| High Fructose Corn Syrup | 24.78% |
| Gelatine 250 Bloom Type A | 5.51% |
| Gellan | 0.33% |
| Sucralose | 0.06% |
| potassium citrate | 1.40% |
| Modified Starch | 2.75% |
| Water | 4.96% |
| Flavour | 0.56% |
| Colour | 0.28% |
| Total: | 100.00% |

The following method was used to prepare the above delivery system. Creatine was added to a mixture of glycerol and propylene glycol, and heated to 40-60° C. The syrups were blended with water and the dry ingredients were mixed into the syrup mixture. The combined mixture was then heated to at least 80° C. Alternatively, the blended dry ingredients can be blended in with simultaneous live steam injection to reach at least 80° C. The solid content was then adjusted by addition of water if necessary to provide a final moisture content of between about 10% to about 30%. At this point, the temperature of the syrup mixture was lowered to between 50° C. and 80° C. and the glycerol-glycol mixture was added. Colour and/or flavouring additives were then added and the delivery system was injection filled into the preformed packaging.

2.8

| Ingredient | % by Weight |
|---|---|
| Glycerol | 27.96% |
| Propylene glycol | 3.44% |
| Potassium hydroxide (45%) | 0.30% |
| Functional ingredient (creatine monohydrate) | 24.07% |
| Corn syrup 63DE | 13.34% |
| High fructose corn syrup | 15.65% |
| Water | 6.30% |
| Potassium phosphate | 0.43% |
| Potassium citrate | 0.96% |
| Sucralose | 0.03% |
| Gelatine | 7.11% |
| Flavour | 0.14% |
| Colour | 0.27% |
| Total: | 100.00% |

2.9

| Ingredient | % by Weight |
|---|---|
| Glycerol | 26.32% |
| Propylene glycol | 3.43% |
| Potassium hydroxide (45%) | 0.23% |
| Functional ingredient (creatine monohydrate) | 24.03% |
| Corn syrup 63DE | 14.24% |
| High fructose corn syrup | 16.72% |
| Water | 4.04% |
| Potassium phosphate | 0.43% |
| Potassium citrate | 0.96% |
| Sucralose | 0.04% |
| Gelatine | 9.15% |
| Flavour | 0.14% |
| Colour | 0.27% |
| Total: | 100.00% |

The delivery systems of Examples 2.8 and 2.9 were prepared as follows. Glycerol and propylene glycol were first blended and the creatine was added. The blend was heated to 45-50° C. The syrups, water, salts and pH modifying agents were mixed and heated to 60-70° C. with constant agitation to dissolve the solids. The gelatine and Sucralose were then incorporated into the syrup mixture using high shear and the temperature was reduced to approximately 50-60° C. Finally, the creatine mixture was added, together with the colour and flavour additives, and blended. The delivery system was moulded using standard techniques.

2.10

| Ingredient | % by Weight |
|---|---|
| Glycerol | 33.0-43.0% |
| High fructose corn syrup | 13.0-19.0% |
| 63 DE corn syrup | 11.0-16.0% |
| Water | 8.0-12.0% |
| Gelatine | 5.0-7.0% |
| Functional ingredient #1* | 3.5-6.5% |
| Functional ingredient #2^{§} | 3.0-5.0% |
| Propylene Glycol | 2.0-3.0% |
| Modified starch | 1.5-3.0% |
| Caffeine | 1.0-2.0% |
| Methylcellulose | 0.8-2.0% |
| Flavour | 0.5-3.0% |
| Colour | 0.01-1.0% |
| Pectin | 0.01-0.3% |
| Artificial sweetener | 0.01-0.2% |
| Vitamin D | 0.005-0.1% |
| Citric acid | 0.0-0.5% |

*calcium carbonate
^{§}Blend of carnitine, ginseng, green tea, taurine, tyrosine and yerbamate

The above formulation was prepared by the following process. The glycerol and propylene glycol were blended. The calcium, methylcellulose and proprietary blend of actives are preblended together then incorporated into the glycerol/propylene glycol and the blend warmed to 40-50° C. When the vitamin D is used in powder form it can be added to the preblend, when used in liquid form, it can be added to the glycerol/propylene glycol prior to adding the dry preblend. The caffeine was dissolved in water heated to between 65° C. and 85° C. The sugar syrups were then incorporated and the temperature adjusted to 60-70° C. The gelatine, pectin, starch and sweetening agents were preblended and introduced into the syrup(s) under shear. The calcium blend was then uniformly blended with the gelatine preparation. Flavour and colour were then added and the whole maintained between 40° C. and 55° C.

### Example 3

Accelerated Shelf-Life Determination

An accelerated shelf life test was conducted on the creatine delivery system prepared by the method described in Example 2.6. Microbial analysis was conducted using approved methods as described in The Compendium of Analytical Methods: HPB Methods for the Microbiological Analysis of Foods (Volume 2) issued by the Health Products and Food Branch of Health Canada. After subjecting samples of the delivery system to a temperature of 35° C. and a relative humidity of 45-55% for a period of 35 days, the samples were tested for the presence of various microorganisms as listed in Table 2. The average water activity of the samples tested was approximately 0.51.

The results, as shown in Table 2, indicate that after a period of 35 days at the above-described conditions, microbial contamination was minimal and well below accepted levels. Based on these results, the delivery system is shown to have a stable shelf life of at least one year from the date of manufacture.

In addition to the above microbial analysis, the creatine level in each sample was determined by HPLC prior to the test and after 35 days. The average creatine content for four samples randomly selected for analysis after 35 days was compared to the average creatine content for three samples taken prior to the shelf life test. The results indicated that levels of creatine monohydrate remained stable in the jujubes after 35 days exposure to the above-described conditions. Prior to the start of the experiment, three jujubes had an average of 13.4% by weight of creatine monohydrate. After 35 days, four jujubes were shown to have an average of 14.2% by weight of creatine monohydrate, which is within the error limits of the analysis performed.

TABLE 2
Microbial Analysis of a Creatine Delivery System
- Accelerated Shelf Life Determination

| | HPB REFERENCE | RESULTS (No. |
|---|---|---|
| TEST CONDUCTED | NUMBER | Colonies/gm product) |
| Total aerobic plate count | MFHPB - 18 | <10 |
| Total coliforms | MFHPB - 34 | <10 |
| E. Coli | MFHPB - 34 | <10 |
| Yeast | MFHPB - 22 | <50 |
| Mould | MFHPB - 22 | <50 |
| Yeast Osmophilic | MFHPB - 22 | <50 |
| Mould Osmophilic | MFHPB - 22 | <50 |
| Staphylococcus aureus | MFHPB - 21 | <25 |
| Salmonella | MFHPB - 20 | not detected |

Water activity: approximately 0.51
Time: 35 days
Temperature: 35° C.
Humidity: 45-55%

### Example 4

Analysis of Water Activity of the Delivery System

Water activity was measured in samples of the delivery system that had been prepared according to the method described in Example 2.6.

The procedure for measuring water activity is based on the fact that the water activity of a sample is equal to the relative humidity created by the sample in a closed environment when in equilibrium. The procedure uses a water activity meter constructed by David Brookman & Associates (DB&A). The DB&A Water Activity Meter uses an Omega Engineering HX92C Relative Humidity indicator to measure 10 the relative humidity within a closed environment containing the sample. The Omega probe converts the relative humidity (R.H.) into milliamperes (ma), where 4 ma equals 0% R.H. and 20 ma equals 100% R.H. The water activity meter is calibrated to 11.3% R.H. using a saturated solution of LiCl and to 75.3% R.H. using a saturated solution of NaCl.

The samples are manually macerated in a plastic bag and then transferred to a 30 ml sample bottle. The bottles are filled with sample to at least 1 cm from the shoulder. The bottles are capped until use and stored at room temperature. Measurements are taken by screwing the sample bottle onto the DB&A meter probe and the bottle probe assembly is maintained in a vertical position in a rack. Measurements are taken at hourly intervals at room temperature (20-22° C.) until such time that successive readings do not vary more than 1%.

Random sampling of the jujubes was conducted. The water activity (a_{w}) was determined to be 0.507, 0.515 and 0.544. These values are well below levels those that favour the growth of microorganisms. It has been shown that microorganisms generally grow best between a_{w} values of 0.995-0.980 and most microbes will cease to grow at a_{w} values less than 0.900.

### Example 5

In vivo Testing I

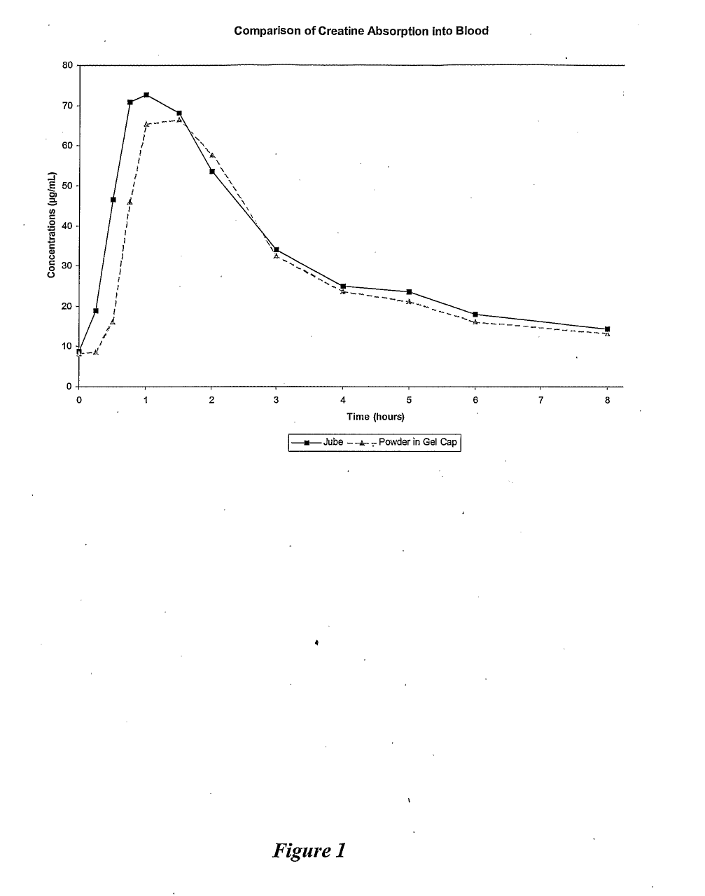

The following example demonstrates the uptake of a functional ingredient (creatine) into the blood after consumption of a delivery system formulated with a matrix as described herein. Serum concentration levels of creatine of subjects who ingested either 3.5 gram of micronized creatine powder in capsule format or 3.5 gram of micronized creatine in jujubes (prepared as described in Example 2.5) were analysed by mass spectroscopy. Seven individuals were enrolled in the test, with an age range between 18 and 50 years. Individuals fasted overnight prior to administration of the creatine. The test protocol was as follows. Individuals were administered jujube containing 3.5 g creatine with 8 oz water. Blood samples were taken every 15 minutes for the first hour, every 30 minutes for the second hour and subsequently at hourly intervals for a total of 8 hours after administration. After sufficient period of time to allow blood creatine levels to return to normal, the subjects were administered 5 capsules containing a total of 3.5 g creatine with 8 oz water. Blood samples were taken at the same time intervals as indicated above. Results are shown in FIG. 1.

The disclosure of all patents, publications, including published patent applications, and database entries referenced in this specification are specifically incorporated by reference in their entirety to the same extent as if each such individual patent, publication, and database entry were specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1. An oral gel delivery system for non-steroidal anti-inflammatory drugs (NSAIDs) comprising one or more NSAIDs substantially uniformly dispersed in a gel matrix, said delivery system having a final moisture content of between about 10% and about 40% by weight and a water activity of less than about 0.9, and said gel matrix comprising:
a) one or more hydrocolloids;
b) one or more sugars, sugar syrups, sugar alcohols, or a combination thereof; and
c) one or more polyhydric alcohols.

2. The oral gel delivery system according to claim 1, wherein said delivery system has a final pH between about 4.5 and about 9.0.

3. The oral gel delivery system according to claim 1 or 2, wherein said one or more NSAIDs comprise up to about 40% by weight of said delivery system.

4. The oral gel delivery system according to any one of claims 1, 2, or 3, wherein said delivery system comprises between about 0.1% and about 17% by weight of said one or more hydrocolloids, between about 10% and about 60% by weight of said one or more sugars, sugar syrups, sugar alcohols, or combination thereof and between about 5% and about 50% by weight of said one or more polyhydric alcohols.

5. The oral gel delivery system according to any one of claims 1, 2, 3 or 4, wherein said one or more hydrocolloids are selected from the group of: gelatine, gellan, pectin, modified starch, cellulose and modified cellulose.

6. The oral gel delivery system according to any one of claims 1, 2, 3, 4 or 5, wherein said one or more sugars, sugar syrups or sugar alcohols are selected from the group of: corn syrup, high fructose corn syrup, maltitol syrup and isomalt syrup.

7. The oral gel delivery system according to any one of claims 1, 2, 3, 4, 5 or 6, wherein said one or more polyhydric alcohols are selected from the group of: glycerol, lower alkyl ester derivatives of glycerol, propylene glycol and short chain polyalkylene glycols.

8. The oral gel delivery system according to any one of claims 1, 2, 3, 4, 5, 6 or 7, wherein said one or more NSAIDs are selected from the group of: aniline derivative NSAIDs, propionic acid derivative NSAIDs and acetic acid derivative NSAIDs.

9. The oral gel delivery system according to any one of claims 1, 2, 3, 4, 5, 6 or 7, wherein said NSAID is a salicylic acid derivative NSAID.

10. The oral gel delivery system according to any one of claims 1, 2, 3, 4, 5, 6 or 7, wherein said NSAID is ibuprofen.

11. The oral gel delivery system according to any one of claims 1, 2, 3, 4, 5, 6 or 7, wherein said NSAID is acetominophen.

12. The oral NSAID gel delivery system according to any one of claims 1, 2, 3, 4, 5, 6 or 7, wherein said NSAID is diclofenac.

13. The oral gel delivery system according to any one of claims 1, 2, 3, 4, 5, 6 or 7, wherein said NSAID is indomethacin.

14. The oral gel delivery system according to any one of claims 1, 2, 3, 4, 5, 6, 7, 8, 9, 10, 11, 12 or 13 further comprising one or more other functional ingredients, wherein the total amount of said one or more NSAIDs and said one or more functional ingredients is less than or equal to 40% by weight of said delivery system.

15. The oral gel delivery system according to claim 14, wherein said one or more other functional ingredients are selected from the group of: anti-inflammatory compounds, antihistamines, decongestants, expectorants, anti-tussives, narcotic analgesics, alkaloids, muscle-relaxants, antacids, anticholinergics, B vitamins, caffeine and phospholipids.

16. The oral gel delivery system according to any one of claims 1, 2, 3, 4, 5, 6, 7, 8, 9, 10, 11, 12, 13, 14 or 15, wherein said delivery system further comprises one or more bioavailability enhancer.

17. An oral gel delivery system for non-steroidal anti-inflammatory drugs (NSAIDs) comprising one or more NSAIDs substantially uniformly dispersed in a gel matrix, said delivery system having a final moisture content of between about 10% and about 30% by weight and a water activity of less than about 0.7, and said gel matrix comprising:
a) one or more hydrocolloids selected from the group of: modified starch, gelatine, gellan, pectin, cellulose and modified cellulose;
b) one or more sugar syrups selected from the group of: corn syrup, high fructose corn syrup, maltitol syrup and isomalt syrup, and
c) one or more polyhydric alcohols selected from the group of: glycerol and propylene glycol.

18. The oral gel delivery system according to claim 17, wherein said delivery system has a final pH between about 6.0 and about 9.0.

19. The oral gel delivery system according to claim 17 or 18, wherein said one or more NSAIDs comprise up to about 40% by weight of said delivery system.

20. The oral gel delivery system according to any one of claims 17, 18 or 19, wherein said delivery system comprises between about 0.1% and about 17% by weight of said one or more hydrocolloids, between about 15% and about 55% by weight of said one or more sugar syrups, and between about 5% and about 50% by weight of said one or more polyhydric alcohols.

21. The oral gel delivery system according to any one of claims 17, 18, 19 or 20, wherein said one or more NSAIDs are selected from the group of: aniline derivative NSAIDs, propionic acid derivative NSAIDs and acetic acid derivative NSAIDs.

22. The oral gel delivery system according to any one of claims 17, 18, 19 or 20, wherein said NSAID is a salicylic acid derivative NSAID.

23. The oral gel delivery system according to any one of claims 17, 18, 19 or 20, wherein said NSAID is ibuprofen.

24. The oral gel delivery system according to any one of claims 17, 18, 19 or 20, wherein said NSAID is acetominophen.

25. The oral gel delivery system according to any one of claims 17, 18, 19 or 20, wherein said NSAID is diclofenac.

26. The oral gel delivery system according to any one of claims 17, 18, 19 or 20, wherein said NSAID is indomethacin.

27. The oral gel delivery system according to any one of claims 17, 18, 19, 20, 21, 22, 23, 24, 25 or 26 further comprising one or more other functional ingredients, wherein the total amount of said one or more NSAIDs and said one or more functional ingredients is less than or equal to 40% by weight of said delivery system.

28. The oral gel delivery system according to claim 27, wherein said one or more other functional ingredients are selected from the group of: anti-inflammatory compounds, antihistamines, decongestants, expectorants, anti-tussives, narcotic analgesics, alkaloids, muscle-relaxants, antacids, anticholinergics, B vitamins, caffeine and phospholipids.

29. The oral gel delivery system according to any one of claims 17, 18, 19, 20, 21, 22, 23, 24, 25, 26, 27 or 28, wherein said delivery system further comprises one or more bioavailability enhancer.

30. Use of a gel matrix comprising:
a) one or more hydrocolloids;
b) one or more sugars, sugar syrups, sugar alcohols, or a combination thereof, and
c) one or more polyhydric alcohols, in the preparation of an oral gel delivery system for non-steroidal anti-inflammatory drugs (NSAIDs), wherein said delivery system comprises one or more NSAIDs substantially uniformly dispersed in said gel matrix, and said delivery system has a final moisture content of between about 10% and about 40% by weight and a water activity of less than about 0.9.

31. The use according to claim 30, wherein said delivery system comprises up to about 40% by weight of said one or more NSAIDs.

32. A process for preparing an oral gel delivery system for non-steroidal anti-inflammatory drugs (NSAIDs), said process comprising the steps of:
(i) preparing a blend of one or more hydrocolloids, one or more sugars, sugar syrups, sugar alcohols, or a combination thereof, and optionally water at a temperature of less than 100° C., wherein said hydrocolloid(s), said sugars, sugar syrups and/or sugar alcohols and said water are in a ratio that will provide a final moisture content to the delivery system of between about 10% and about 40% by weight;
(ii) reducing the temperature of said blend to between about 50° C. and about 80° C.;
(iii) dispersing one or more NSAIDs in a solvent comprising one or more polyhydric alcohols at a temperature at or below about 70° C. to provide a solvent mixture;
(iv) combining said blend from step (ii) with said solvent mixture to provide a gel matrix, and
(v) moulding said gel matrix to provide said oral gel delivery system.

33. The process according to claim 32, wherein the amount of said one or more NSAIDs dispersed in said solvent in step (iii) provides up to 40% by weight of said NSAID(s) in the final delivery system.

34. The process according to claim 32 or 33, wherein preparing said blend in step
(i) is at a temperature between about 60° C. and about 80° C.

35. The process according to any one of claims 32, 33 or 34, wherein dispersing said one or more NSAIDs in said solvent in step (iii) is at a temperature below about 50° C.

36. An oral gel delivery system for non-steroidal anti-inflammatory drugs NSAIDs) prepared by the process of any one of claims 32, 33, 34 or 35.

37. Use of the oral gel delivery system according to any one of claims 1, 2, 3, 4, 5, 6, 7, 8, 9, 10, 11, 12, 13, 14, 15 or 16 to deliver an effective amount of one or more non-steroidal anti-inflammatory drugs (NSAIDs) to an animal in need thereof.

38. The use according to claim 37, wherein said one or more NSAIDs are for reducing and/or preventing pain, inflammation, fever or a combination thereof in said animal.

39. The use according to claim 37 or 38, wherein said animal is a human.

40. Use of the oral gel delivery system according to any one of claims 17, 18, 19, 20, 21, 22, 23, 24, 25, 26, 27, 28 or 29 to deliver an effective amount of one or more non-steroidal anti-inflammatory drugs (NSAIDs) to an animal in need thereof.

41. The use according to claim 40, wherein said one or more NSAIDs are for reducing and/or preventing pain, inflammation, fever or a combination thereof in said animal.

42. The use according to claim 40 or 41, wherein said animal is a human.

43. A kit for the delivery of one or more non-steroidal anti-inflammatory drugs (NSAIDs) to an animal comprising one or more units of the oral gel delivery system according to any one of claims 1, 2, 3, 4, 5, 6, 7, 8, 9, 10, 11, 12, 13, 14, 15 or 16 and optionally instructions for use.

44. A kit for the delivery of one or more non-steroidal anti-inflammatory drugs (NSAIDs) to an animal comprising one or more units of the oral gel delivery system according to any one of claims 17, 18, 19, 20, 21, 22, 23, 24, 25, 26, 27, 28 or 29 and optionally instructions for use.

45. The kit according to claim 43 or 44, wherein said animal is a human.
